(19) United States
(12) Patent Application Publication
(10) Pub. No.: US 2015/0030676 A1
(43) Pub. Date: Jan. 29, 2015

(54) STABLIZED MODIFIED RELEASE FOLIC ACID DERIVATIVE COMPOSITION, ITS THERAPEUTIC USE AND METHODS OF MANUFACTURE

(71) Applicant: APTALIS PHARMATECH, INC., Vandalia, OH (US)

(72) Inventors: Gopi M. VENKATESH, Vandalia, OH (US); Micael GUILLOT, Lansdale, PA (US); Jin-Wang LAI, Springboro, OH (US); Michael GOSSELIN, Springboro, OH (US); Nicole A. BEINBORN, Dayton, OH (US)

(21) Appl. No.: 14/445,949

(22) Filed: Jul. 29, 2014

Related U.S. Application Data
(60) Provisional application No. 61/859,627, filed on Jul. 29, 2013.

(51) Int. Cl. A61K 9/28; A61K 9/20; A61K 31/519

(52) U.S. Cl. CPC A61K 9/2866; A61K 31/519; A61K 9/2027; A61K 9/2893; A61K 9/2886; A61K 9/2054; A61K 9/2013; A61K 9/282; A61K 9/2853; A61K 9/2095
USPC 424/465; 514/249; 427/2.21; 264/122

(57) ABSTRACT

This invention relates to an oral stabilized modified release pharmaceutical dosage form containing L-methylfolate calcium, which is primarily absorbed from proximal small intestine via a saturable human proton-coupled folate transporter (h-PCFT) mediated transport intended as monotherapy for the treatment of patients with MDDs and/or diagnosed with dysthymia, schizophrenia, or degenerative dementia of the Alzheimer type.

### REFERENCE TO PRIOR APPLICATION

This application claims the benefit of U.S. Provisional Application No. 61/859,627, filed Jul. 29, 2013, the disclosure of which is hereby incorporated by reference herein in its entirety.

### FIELD OF THE INVENTION

This invention relates to a stabilized, pharmaceutical composition comprising a folic acid derivative including a pharmaceutically acceptable salt thereof. The invention also relates to a therapeutic use of the composition, particularly for treating patients with major depressive disorder (MDD), diabetic peripheral neuropathy or schizophrenia. Furthermore, the invention relates a method of manufacture of the stabilized, pharmaceutical composition.

### BACKGROUND OF THE INVENTION

Folic acid (pteroylglutamic acid) I, which is not synthesized by the cells of

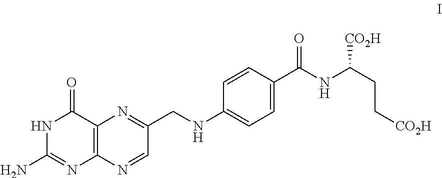 [SMILES: Nc1nc2ncc(CNc3ccc(C(=O)N[C@@H](CCC(=O)O)C(=O)O)cc3)nc2c(=O)[nH]1]

mammals, is of particular biological importance due to the activity of derivatives thereof, i.e., folates. For example, folic acid, which itself is biologically not active, is used for food fortification given its metabolism to folates that can prevent the incidence of neural birth defects. The derivatives of folic acid, including tetrahydrofolic acid, 5-methyltetrahydrofolic acid (5-MTHF), 5-formyltetrahydrofolic acid, and their salts, are a group of substances pertaining to the vitamin B complex. Natural food folates are a mixture of reduced forms of the vitamin, most predominantly, 5-methyltetrahydrofolate and usually in the polyglutamylated form containing variable number of glutamate residues. 5-MTHF is considered to a better alternative to folic acid as it is more likely to minimize the symptoms of B12 deficiency in older populations. L-methylfolate, or 6(S)-5-methyltetrahydrofolate (6(S)-5-MTHF), is the primary

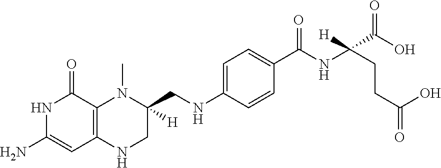 [SMILES: CN1c2c(cc(N)[nH]c2=O)NC[C@@H]1CNc1ccc(C(=O)N[C@@H](CCC(=O)O)C(=O)O)cc1]

biologically active isomer of folate in circulation. Reduced forms of folates serve as single carbon unit acceptors or donors, a reaction collectively called ‘single carbon metabolism’ In particular, L-methylfolate is a critical element in the one carbon unit cycle, involved in neurotransmitter synthesis, nucleic acid methylation, and neuronal plasma methylation. 5-MTHF is also the form which is transported across membranes into peripheral tissues, particularly across the blood brain barrier, in contrast to folic acid which does not. Folates also act as coenzyme substrates in many reactions of amino acids and nucleotides. In cells, 6(S)-5-MTHF is used in the methylation of homocysteine to form methionine and tetrahydrofolate (THF). THF is also used as the immediate acceptor of one carbon unit for the synthesis of thymidine-DNA, purine-RNA and purine-DNA.

All folate compounds are sensitive and easily degraded under high temperatures, air or oxygen, light, low pH, and reducing agents. Antioxidants such as ascorbates have shown to minimize such degradations during processing and storage (Nguyen, M. T., Indrawati, & Hendrickx, M. (2003), Journal Agricultural Food Chemistry, 51, 3352-3357; Indrawati, Arroqui, C., Messagie, I., Nguyen, M. T., Loey, A. V., & Henderickx, M. (2004), Journal Agricultural Food Chemistry, 52, 485-492). It is reported that L-5-MTHF-Ca in microencapsulated form, preferably with an ascorbate as an antioxidant, protects the methylfolate from degradation during processing, thereby resulting in a long term stability in a variety of foodstuffs.

A. R. Muller et al. disclose in U.S. Pat. No. 6,011,040, U.S. Pat. No. 6,271,374, U.S. Pat. No. 6,441,168 and U.S. Pat. No. 6,995,158 the preparation of highly crystalline pentahydrate of calcium salt of 5-methyl-(6S)-tetrahydrofolic acid. The references however do not described stabile modified release compositions comprising the highly crystalline pentahydrate of calcium salt of 5-methyl-(6S)-tetrahydrofolic acid.

C. L. Grazie disclosed in U.S. Pat. No. 5,059,595 and U.S. Pat. No. 5,538,734 the preparation of 5-methyltetrahydrofolate controlled release (CR) gastroresistant tablets with an average release time of 20 to 60 minutes comprising 5, 15, 20, 25, 40, 100, or 200 mg of MTHF, formyl-tetrahydrofolic acid (FTHF) or their salt. The therapeutically positive effects of daily dosing of 50 mg MTHF CR tablets (complete release within 60 minutes) in comparison to the 50 mg immediate release (IR) MTHF tablets in randomized groups each of 30 depressed patients for 90 days, as measured by appropriate clinical end points, were compared after 21, 45 or 90 days. No stability data was disclosed regarding the CR tablets.

The intestinal absorption of dietary medical food, methylfolates is a two-step process involving (a) hydrolysis of folate polyglutamates principally at a pH of 6.5 to the corresponding monoglutamyl derivatives and (b) saturable transport, via a proton-coupled co-transport mechanism, into the enterocyte. In humans, the proton coupled folate transporter (h-PCFT), a protein with 459 amino acids, which actively transports methyl folate across the blood-brain barrier is most abundantly expressed in the upper small intestine and less in the lower small intestine, localizing at the brush border membrane of epithelial cells. The proton coupled folate transporter has a high affinity for folate and its analogs with a Michaelis constant (Kₘ) of a 1.7 μM at pH 5.0-5.5. A loss of PCFT function due to a homozygous mutation in its gene has been indicated to be responsible for hereditary folate malabsorption (Yuasa et al., 2009. Molecular and functional characteristics of proton-coupled folate transporter, J. Pharma. Sci. 98(5), 1608-1616).

L-isomer of 5-MTHF is a water soluble compound that is primarily excreted via the kidneys. In the body and by first pass metabolism, folic acid and folinic acid, which have structures similar to 5-MTHF, are primarily reduced to form 5-MTHF. L-methylfolate is formed from the enzymatic reduction of either dietary dihydrofolate or synthetic folic acid with final step regulated by methylenetetrahydrofolate reductase (MTHFR). Red blood cells appear to be the storage depot for folate, as red cell levels remain elevated for periods in excess of 40 days following discontinuation of supplementation.

It has been generally believed that there is some form of association between folate-deficiency states and depression (V. Herbert, Experimental nutritional folate deficiency in man. Trans Assoc Am Phys 75 (1961), p. 307; M. W. P. Carney, Serum folate values in 423 psychiatric patients. Br Med J 4 (1967), p. 512; E. H. Reynolds, J. M. Preece, J. Bailey and A. Coppen, Folate deficiency in depressive illness. Br. J. Psychiatry 117 (1970), p. 287), which in turn helps to explain prior observations on the myriad neuropsychiatric presentations of megaloblastic anemia (S. D. Shorvon, M. W. P. Carney, I. Chanarin and E. H. Reynolds, The neuropsychiatry of megaloblastic anaemia. Br Med J 281 (1980), p. 1036). Recently, the relevance of folate in other medical conditions, in particular neural tube defects (MRC Vitamin Study Research Group, Prevention of neural tube defects: results of the Medical Research Council Vitamin Study. Lancet 338 (1991), p. 131) and cardiovascular disease (E. B. Rimm, W. C. Willett, F. B. Hu et al., Folate and vitamin B6 from diet and supplements in relation to risk of coronary heart disease among women. JAMA 279 (1998), p. 359), and potential antidepressant efficacy of agents marketed as dietary supplements or “nutraceuticals,” (D. Mischoulon, Herbal remedies for mental illness. Psychiatr Clin North Am Annu Drug Ther 6 (1999), p. 1; A. Fugh-Berman and J. M. Cott, Dietary supplements and natural products as psychotherapeutic agents. Psychosom Med 61 (1999), p. 712) such as S-adenosyl-methionine (SAMe), hypericum perforatum (St. John's wort), and omega-3-fatty acids, has been increasingly recognized. Thus, the field has gradually moved toward researching the impact of folate deficiency, replacement and supplementation on the course and management of a number of disorders; particularly depressive disorders, in particular MDD (American Psychiatric Association. Diagnostic and statistical manual of mental disorders, 4th ed. Washington, D.C.: American Psychiatric Association, 1994), and putative roles of folate in central-nervous-system function (T. Bottiglieri, Folate, vitamin B12, and neuropsychiatric disorders. Nutr Rev 54 (1997), p. 382; J. E. Alpert and M. Fava, Nutrition and depression: the role of folate. Nutr Rev 55 (1997), p. 14; B. R. Hutto, Folate and cobalamin in psychiatric illness. Compr Psychiatry 38 (1997), p. 305).

The importance of examining the impact of folate deficiency, replacement and supplementation on the course and management depressive disorders is due to the fact that it is estimated that more than 19 million Americans over the age of 18 years experience a depressive illness each year, and 15% of those who suffer from depression will attempt suicide. MDD is a debilitating illness affecting 7% to 12% of men and 20% to 25% of women. It is usually a recurrent illness, with up to 30% of patients experiencing a depressive episode lasting over 2 years. The U.S. MDD therapeutics market in 2010 was $7.7B and is expected to remain fairly stabile through 2020. Although the goal in treating MDD is full remission, however for most patients, remission is the exception rather than the rule. An initial antidepressant trial is effective at achieving remission for ˜30% of patients when prescribed as monotherapy, with the majority of patients returning as either partial or non-responders. Switching antidepressants or adding augmentation agents are standard therapeutic options used to achieve and maintain remission. While significant advances in the treatment of depression have been made in the past decade, as many as 29% to 46% of patients with depression taking an anti-depressant are still partially or totally resistant to the treatment.

Adequate levels of central nervous system (CNS) folate are likely essential for a patient to fully recover from a depressive episode. Suboptimal serum and red blood cell (RBC) folate levels have been associated with a poorer response to antidepressant therapy, a greater severity of symptoms, later onset of clinical improvement, and overall treatment resistance. Lower systemic levels of folate can also result from poor dietary intake, diabetes, various gastrointestinal disorders, hypothyroidism, renal failure, nicotine dependence, alcoholism. This lower folate level is associated with a particular genetic polymorphism prevalent in 50% of the United States population, and up to 70% of depressed patients (Andrew Farah, The Role of L-methylfolate in Depressive Disorders. CNS Spectrums 2009; 16:1(Suppl 2):1-7). The genetic polymorphism called MTHFR polymorphism limits the body's ability to reduce dietary folate or folic acid into L-methylfolate. Two variant mutations of the MTHFR enzyme, a C677T genotype, which is more common, and a homozygous T677T genotype, which is the more severe of the two forms, are exhibited in depressed population. Approximately 15% of patients with depression exhibit the homozygous T677T genotype, supporting the link between folate deficiency and depression.

L-methylfolate is used by the body in the nutritional management of neurotransmitters (necessary chemicals) that affect mood. The importance of L-methylfolate in depression is that it, unlike folic acid, can cross the blood brain barrier to augment the activity of antidepressants by acting as a trimonoamine modulator. A reduction in MTHFR activity leads to a decrease in the monoamine neurotransmitter pool, thereby rendering anti-depressant agents ineffective.

Medication strategies for treating depression involve a number of strategies, including augmenting the treatment regimen with a non-antidepressant agent, such as L-methylfolate, to increase rates of response and decrease the risk for relapse (T. Bottiglieri, P. Godfrey et al., Lancet. 1990 Dec. 22-29; 336:1579-1580; M. Passeri M et al., Aging (Milano). 1993 February; 5(1):63-71; G P. Guaraldi et al., Ann Clin Psychiatry. 1993 June; 5(2):101-105; M. Fava, J Clin Psychiatry. 2001; 62 Suppl 18:4-11; M. Fava, J Clin Psychiatry. 2007; 68 Suppl 10:4-7; D W. Morris et al., J Altern Complement Med. 2008 April; 14(3):277-285; A. Farah, CNS Spectr. 2009 January; 14(1 Suppl 2):2-7; L D. Ginsberg et al., Innov Clin Neurosci. 2011 January; 8(1):19-28; M. Fava, D. Mischoulon; J. Clin Psychiatry. 2009; 70 (Suppl 5) 12-17; G I Papakostas et al., J. Clin Psychiatry. 2009; 70 (Suppl 6) 16-25). Deplin®, a medical food marketed for patients with MDD since 2007, has established itself as a safe and well tolerated in its use for treating depression. L-methylfolate is water soluble and has low potential for drug interactions. Its side effects and discontinuation due to adverse events is similar to placebo. Based on multicenter sequential parallel comparison design trials to investigate the effect of L-methylfolate augmentation in the treatment of MDD in patients who had a partial response or no response to selective serotonin reuptake inhibitors (SSRIs), the adjunctive L-methylfolate at 15 mg/day has been shown to be an effective, safe, and relatively well tolerated treatment strategy for patients with MDD who have a partial response or no response to SSRIs. The data showed that 32% of the patients who received adjunctive therapy with 15 mg of Deplin® combined with an SSRI responded after 30 days of treatment compared to 14.6% of patients who received SSRI with placebo (p=0.04) (G I Papakostas et al., Am. J. Psychiatry. 2012 Dec. 1; 169: 1267-1274).

Antidepressant and placebo response rates observed in multiple studies (n=34,780; 248 drug-placebo pair-wise comparisons) were analyzed to be 53.4 and 36.6% (p<0.05), respectively (G I. Papakostas, J Clin Psychiatry. 2009; 70 Suppl 5:18-22). Suboptimal folate levels may increase the risk of depression and reduce the activity of antidepressants such as serotonin reuptake inhibitors and monoamine oxidase inhibitors. The 223 patient study was presented by George I. Papakostas at the NCDEU 51st Annual Meeting. Honolulu (HI) the week of June 13^{th}, 2011 (George I. Papakostas, NCDEU 51st Annual Meeting. Honolulu, Hi. 13 Jun. 2011. Scientific and Clinical Report Presentation). New findings from a multi-center, randomized, placebo-controlled clinical study of Deplin® 15 mg (L-methylfolate) added to commonly prescribed antidepressants known as selective serotonin reuptake inhibitors (SSRIs) showed that all patients who achieved remission at 30 days using Deplin® 15 mg adjuvant therapy, and who chose to enter a 12 month maintenance phase, maintained their remission after a year of treatment. The study conclusions support the growing body of evidence for the metabolic management of MDD with Deplin®, a medical food, administered in combination with antidepressants.

DEPLIN is an immediate release (IR) prescription medical food is sold at dosage strengths of 7.5 mg and 15 mg by PAMLAB® LLC as a dietary supplement for the management of suboptimal folate levels in depressed patients or hyperhomocysteinemia in schizophrenia patients. The highly crystalline pentahydrate of calcium salt of 5-methyl-(6S)-tetrahydrofolic acid disclosed in disclose in U.S. patents (U.S. Pat. No. 6,011,040, U.S. Pat. No. 6,271,374, U.S. Pat. No. 6,441,168, and U.S. Pat. No. 6,995,158 is the salt form is used in the immediate release tablet formulation marketed. While the salt form is noted to be stabile, Deplin's shelf-life, which has been established based on stability testing at 20° C./60% RH (long-term stability condition), is limited due to its instability. The moisture content when prepared is 4% or higher, and the total specified impurities is 3% or higher. In order to address the potency (stability) loss of the product that is due to oxidative/hydrolytic degradation of L-methylfolate, each tablet of 7.5 or 15 mg Deplin® is designed to have a potency of 130% by weight of the label claim in order to ensure that the potency of the tablet remains above at least 90% of the label claim during shelf-life. Thus, Deplin® does not represent a stabilized pharmaceutical L-methylfolate composition. Furthermore, Deplin® does not represent a stabilized pharmaceutical L-methylfolate composition that addresses the short plasma elimination half life of L-methylfolate or targets its delivery to the upper small intestine where the human proton coupled folate transporter (h-PCFT) is most abundantly expressed.

### SUMMARY OF THE INVENTION

This invention relates to a stabilized modified release pharmaceutical composition comprising a folic acid derivative or a pharmaceutically acceptable salt thereof, such as L-methylfolate (e.g., tetrahydrofolic acid or its derivative, 5-methyl tetrahydrofolic acid, 5-formyl tetrahydrofolic acid, or their isomers). The invention is also directed to a composition that exhibits target in vitro drug release profile and/or pharmacokinetic (PK) profile suitable for a once- or twice-daily dosing regimen. Furthermore, the invention is directed to methods of making and using such a composition for the treatment of patients with MDDs, diagnosed with dysthymia, schizophrenia or degenerative dementia of the Alzheimer type, to prevent neural defects, to prevent cardiovascular disorders or to exclude a health risk (masking pernicious anemia, irreversible neuropathy).

### BRIEF DESCRIPTION OF THE DRAWINGS

FIG. 1 shows the release of pentahydrate of calcium salt of 5-methyl-(6S)-tetrahydrofolic acid 6(S)-5-methyl tetrahydrofolate (6(S)-5-MTHF of calcium) from delayed release (DR) minitablets coated with a talc-containing hypromellose phthalate membrane of Example 2 at 13.8%, 22.5%, 25%, 27.5%, and 30% by weight in comparison to that from minitablets with a 14% membrane coating having no talc.

FIG. 2 shows the 6(S)-5-MTHF of calcium release from 13.8% or 26.5% DR coated minitablets or timed pulsatile release (TPR) minitablets with a TPR coating (1.3% TPR coating disposed over 13.8% DR coating), (1% TPR coating over 26.5% DR coating), or (2.5% or 5% TPR coating over 30% DR coating), both DR and TPR coating membranes containing talc.

FIG. 3 shows the 6(S)-5-MTHF of calcium release from DR minitablets having a 14% non-talc DR membrane coating or 26.5% talc-containing DR membrane coating and TPR minitablets with a TPR coating (1%, 2% or 3% TPR coating over 14% DR coating, both being non-talc membrane coatings) or (1% TPR coating over 26.5% or 30% DR coating, both DR and TPR coating membranes containing no talc).

FIG. 4 shows the physical stability of in vitro 6(S)-5-MTHF of calcium release from 50 mg MR tablets of Example 3 when stored in induction-sealed HDPE bottles at 40° C./75% RH for 6 months or at 25° C./60% RH for 12 months.

FIG. 5 shows the pharmacokinetics profiles of 6(S)-5-MTHF of calcium observed upon a single oral administration of 50 mg IR tablets or 50 mg MR Tablets in healthy volunteers under fasted and fed state [(open circle)—IR tablets, fasted state; (filled circle)—IR Tablets, fed state; (open triangle)—MR tablets, fasted state; (filled triangle)—MR Tablets, fed state].

FIG. 6 shows the pharmacokinetics profiles of 6(S)-5-MTHF of calcium observed upon a single oral administration of 19.5 mg or 50 mg IR tablets or 20 mg or 50 mg 6(S)-5-MTHF of calcium MR tablets in randomized, cross-over, dose-dependent parallel groups of healthy, adult volunteers [(open circle)—19.5 mg IR tablets; (open triangle—50 mg IR Tablets; (filled circle)—20 mg MR tablets; (filled triangle)—50 mg MR Tablets].

FIG. 7 shows the pharmacokinetics profiles of 6(S)-5-MTHF of calcium on dosing day 1 and 7 observed for 20 mg and 50 mg MR tablets in sequenced groups of healthy, adult volunteers [(open circle)—20 mg MR tablets on day 1; (open triangle—20 mg MR tablets on day 7; (filled circle)—50 mg MR tablets on day 1; and (filled triangle)—50 mg MR Tablets on day 7].

FIG. 8 shows the in vitro release profiles of 6(S)-5-MTHF of calcium dosage forms: MR capsules, 20 and 50 mg (left curves) and 19.5 mg Deplin®, 50 mg Deplin®-like IR tablets, and MR tablets, 20 mg and 50 mg (right curves).

FIG. 9 shows the in vitro dissolution profiles of 6(S)-5-MTHF of calcium observed for 20 mg and 40 mg MR tablets.

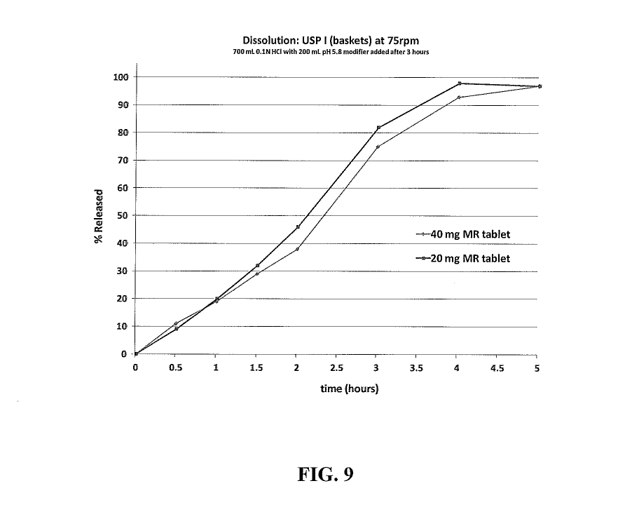

### DETAILED DESCRIPTION OF THE INVENTION

As used above, and throughout the description of the invention, the following terms, unless otherwise indicated, shall be understood to have the following meanings:

The term “drug”, “active”, “active agent”, or “active pharmaceutical ingredient” as used herein includes a pharmaceutically acceptable and therapeutically effective base compound, a pharmaceutically acceptable salt thereof, stereoisomer thereof or mixture of stereoisomers, solvate (including hydrate) thereof, polymorph thereof, and/or prodrug thereof.

Medical foods are foods that are specially formulated and intended for the dietary management of a disease that has distinctive nutritional needs that cannot be met by normal diet alone. They were defined in the Food and Drug Administration's 1988 Orphan Drug Act Amendments and are subject to the general food and safety labeling requirements of the Federal Food, Drug, and Cosmetic Act. In order to be considered a medical food the product must, at a minimum:
be a food for oral ingestion or tube feeding (nasogastric tube)
be labeled for the dietary management of a specific medical disorder, disease or condition for which there are distinctive nutritional requirements, and
be intended to be used under medical supervision.

The term “salts” refers to the product formed by the reaction of a suitable inorganic or organic acid with the “free base” form of the drug. Suitable acids include those having sufficient acidity to form a stabile salt, for example acids with low toxicity, such as the salts approved for use in humans or animals. Non-limiting examples of acids that may be used to form salts include inorganic acids, e.g., HF, HCl, HBr, HI, H₂SO₄, H₃PO₄; non-limiting examples of organic acids include organic sulfonic acids, carboxylic acids, amino acids. Other suitable salts can be found in, e.g., S. M. Birge et al., J. Pharm. Sci., 1977, 66, pp. 1-19 (herein incorporated by reference for all purposes). In most embodiments, “salts” refers to salts that are pharmaceutically (biologically compatible) acceptable, i.e., non-toxic, particularly for mammalian cells. The salts of drugs useful in the invention may be crystalline or amorphous, or mixtures of different crystalline forms and/or mixtures of crystalline and amorphous forms.

The term “prodrug” means a form of the compound of formula I suitable for administration to a patient without undue toxicity, irritation, allergic response, and the like, and effective for their intended use, including ketal, ester and zwitterionic forms. A prodrug is transformed in vivo to yield the active drug product, for example by hydrolysis in blood. A thorough discussion is provided in T. Higuchi and V. Stella, Pro-drugs as Novel Delivery Systems, Vol. 14 of the A. C. S. Symposium Series, and in Edward B. Roche, ed., Bioreversible Carriers in Drug Design, American Pharmaceutical Association and Pergamon Press, 1987, both of which are incorporated herein by reference.

As used herein, the term “pharmaceutically acceptable excipient” encompasses a dissolution rate controlling matrix-forming polymer”, “bioadhesive polymer”, “filler/diluent”, “sugar”, “antioxidant”, “polymeric binder”, “disintegrant”, “lubricant”, “glidant”, “dissolution rate controlling coating polymer, “optional plasticizer” typically used in the coating of drug containing particles, minitablets or tablets, which are normally utilized in the preparation of pharmaceutical compositions, such as modified release drug delivery systems for administration in mammals for the treatment of an inflammation, disease or disorder.

The term, ‘pharmaceutically acceptable excipient’ as used in certain embodiments of the invention has normally multiple functionalities. For example, hypromellose (hydroxypropyl methyl cellulose) with a low viscosity ((e.g., METHOCEL E5) is a polymer binder that can be used in combination with another hypromellose with a higher viscosity (e.g., METHOCEL E4M) that acts as a dissolution rate controlling polymer.

The term, “hydrophilic, dissolution rate controlling, matrix-forming polymer”, as used in certain embodiments of the invention swells on exposure to water or body fluid forming a swollen polymer matrix in which the active pharmaceutical ingredient such as L-methylfolate of calcium is embedded. The drug dissolved in the process diffuses through the swollen gel into the desired gastrointestinal environment. Non-limiting examples of dissolution rate controlling swelling/gelling polymers include hydrophilic hydroxypropyl cellulose, hypromellose (hydroxypropyl methyl cellulose) or polyethylene oxide of different viscosities and mixtures thereof.

The term, ‘pharmaceutically acceptable excipient/dissolution rate controlling polymer’ as used in certain other embodiments of the invention refers to a “bioadhesive polymer”, which swells on exposure to water or body fluid and adheres to the surface such as mucosa of the gastrointestinal tract, thereby increasing the residence time of the dosage form or drug-containing particles. Non-limiting examples of dissolution rate controlling bioadhesive polymers include low substituted hydroxypropyl cellulose of different substitutions, crosslinked polyacrylic acids of different crosslinking densities, commercially known as CARBOPOL 971P or G-71, and polyethylene oxide, POLYOX of different molecular weights, and mixtures thereof.

The term, ‘pharmaceutically acceptable excipient’ as used in certain embodiments of the invention refers to a “filler/diluent” selected from the group consisting of sugars (for example, either a sugar alcohol, such as mannitol, sorbitol, xylitol, or a saccharide, such as lactose, fructose), dicalcium phosphate dihydrate, calcium sulfate, silicified microcrystalline cellulose (PROSOLV SMCC 90 or PROSOLV SMCC 90HD) and mixtures thereof.

The term, ‘pharmaceutically acceptable excipient’ as used in some embodiments of the invention refers to a “sugar” selected from the group consisting of a sugar alcohol, such as mannitol, sorbitol, xylitol, or a saccharide, such as lactose, sucrose, fructose.

The term, ‘pharmaceutically acceptable excipient’ as used in certain other embodiments of the invention refers to an “antioxidant” selected from the group consisting of ascorbic acid or sodium ascorbate, anhydrous citric acid, glutathione, vitamin C, vitamin A, and vitamin E.

The term, ‘pharmaceutically acceptable excipient/dissolution rate controlling, coating polymer’ as used in certain other embodiments of the invention refers to a water soluble, water insoluble, enteric polymer and such a coating layer optionally includes a plasticizer.

As used herein, the term “controlled-release” coating encompasses coatings that delay, sustain, prevent, extend, modify, and/or otherwise prolong the release of a drug from a particle coated with a controlled-release coating. The term “controlled-release” encompasses “sustained-release”, “modified-release”, “extended-release” and “timed, pulsatile release”. Thus, a “controlled-release coating” encompasses a sustained release coating, timed, pulsatile release coating or “lag-time” coating.

The term “pH sensitive” as used herein refers to polymers that exhibit pH dependent solubility, i.e., either gastrosoluble (soluble in the acidic pH range of 1 to 5) or enterosoluble (soluble in the alkaline pH range of 6 to 10). The potential of bioadhesive polymers in holding onto gastrointestinal mucosa due to interfacial forces, thereby leading to a significantly prolonged residence time of sustained release delivery systems would offer various advantages such as extended release characteristics, especially for those drugs with short absorption windows.

The term “enteric polymer”, as used herein, refers to a pH sensitive polymer that is resistant to gastric juice (i.e., relatively insoluble at the low pH levels found in the stomach), and that dissolves at the higher pH levels found in the intestinal tract.

As used herein, the term “immediate release” (IR; in reference to a pharmaceutical composition that can be a dosage form or a component of a dosage form), refers to a pharmaceutical composition that releases greater than or equal to about 50% of the active, in another embodiment greater than about 75% of the active, in another embodiment greater than about 90% of the active, and in other embodiments greater than about 95% of the active within about 60 minutes, following administration of the dosage form.

The term “immediate release particle” refers broadly to an active agent-containing crystal, bead, pellet or minitablet that exhibits “immediate release” properties as described herein.

The term “sustained release (SR) coating” refers broadly to an SR coating comprising a water-insoluble polymer, a fatty acid, a fatty alcohol, a fatty acid ester, as described herein, disposed directly over a active agent-containing particle (e.g., crystal, bead, pellet, minitablet, or tablet) or alternately over the protective seal- or under-coat (seal coat or sealant) disposed over a active agent-containing particle. The outer coating such as a controlled release coating disposed over active agent containing particles (e.g., crystals, beads, pellets, or minitablets) or the protective seal coating disposed over the polymer matrix based MR tablet, which substantially stabilizes active agent during processing, packaging, and storage, is referred to as “stabilizing coating”.

The term “lag-time coating” or “TPR coating” refers to a controlled-release coating comprising the combination of water-insoluble and enteric polymers as used herein. A TPR coating by itself provides an immediate release pulse of the drug, or a sustained drug-release profile after a pre-determined lag time. The term “lag-time coating” or “TPR coating” also refers to a bilayer controlled-release coating, wherein a first layer or coating comprises an enteric polymer and a second layer or coating comprises the combination of water-insoluble and enteric polymers as disclosed in U.S. Pat. No. 6,627,223. The term “lag-time (TPR) bead” or “lag-time particle” refers broadly to a bead or particle comprising a TPR coating or a bilayer coating, as described herein or in U.S. Pat. No. 6,627,223, disposed over methylfolate-containing crystal, bead, pellet or minitablet. The term “lag-time” as used herein refers to a time period wherein less than about 10% of the active is released from a pharmaceutical composition after ingestion of the pharmaceutical composition (or a dosage form comprising the pharmaceutical composition), or after exposure of the pharmaceutical composition, or dosage form comprising the pharmaceutical composition, to simulated body fluid(s), for example evaluated with a United States Pharmacopeia (USP) apparatus using a two-stage dissolution medium (first hour in 700 mL of 0.1N HCl at 37° C. followed by dissolution testing at pH=5.8 obtained by the addition of 200 mL of a pH modifier).

The term “disposed over”, in reference to a coating over a substrate, refers to the relative location of the coating in reference to the substrate, but does not require that the coating be in direct contact with the substrate. For example, a first coating “disposed over” a substrate can be in direct contact with the substrate, or one or more intervening materials or coatings can be interposed between the first coating and the substrate. In other words, for example, a SR coating disposed over a drug-containing core can refer to a SR coating deposited directly over the drug-containing core or acid crystal or acid-containing core, or can refer to a DR or SR coating deposited onto a protective seal coating deposited on the drug-containing core.

The term “sealant layer” or “protective seal or under-coating” refers to a protective membrane disposed over a drug-containing core particle or a functional polymer coating. The sealant layer protects the particle from abrasion and attrition during handling, and/or minimizes static during processing. In the claimed invention; the sealant coating has the stabilizing effect.

The term “dissolution rate-controlling matrix” or “delayed release particle” refers broadly to a solid dosage form (e.g. tablet) comprising dissolution rate-controlling matrix material such as a pharmaceutically acceptable water-insoluble, swelling, gelling and/or eroding polymer or a fatty acid, a fatty alcohol, a fatty acid ester, as described herein.

The term “stabilized dosage form” refers broadly to a solid dosage (e.g. tablet, minitablet, microtablet, drug-containing particle coated with at least one protective coating layer comprising a hydrophilic polymer or a hydrophobic wax and packaged for storage in induction-sealed glass or HDPE bottles with 2-in-1 desiccants and/or oxygen-scavengers or Aclar, cold form or Alu-Alu blisters so that the MR dosage forms exhibit significantly improved stability profiles compared to currently marketed IR products.

The term “substantially disintegrates” refers to a level of disintegration amounting to disintegration of at least about 50%, at least about 60%, at least about 70%, at least about 80%, at least about 90%, or about 100% disintegration. The term “disintegration” is distinguished from the term “dissolution”, in that “disintegration” refers to the breaking up of or loss of structural cohesion of the constituent particles comprising a tablet, whereas “dissolution” refers to the solubilization of a solid (particularly drug) in a liquid (e.g., the solubilization of a drug in solvents or gastrointestinal fluids).

The term “water-insoluble polymer” refers to a polymer that is insoluble or very sparingly soluble in aqueous media, independent of gastrointestinal pH, or over a broad pH range (e.g., pH<1 to pH 8). A polymer other than an enteric (enterosoluble) or gastrosoluble (reverse enteric) polymer that may swell but does not dissolve in aqueous media is considered “water-insoluble,” as used herein. Thus, as used herein, the term “water-insoluble polymer” refers only to a polymer which is insoluble in the physiologically relevant pH media, i.e., insoluble in the aqueous media at pH<1 to pH 8.

The term “enteric polymer” refers to a polymer that is soluble (i.e., a significant amount dissolves) under intestinal conditions; i.e., in aqueous media under ˜neutral to alkaline conditions and insoluble under acidic conditions (i.e., low pH).

The term “reverse enteric polymer” or “gastrosoluble polymer” refers to a polymer that is soluble under acidic conditions and insoluble under neutral (as in water) and alkaline conditions.

The terms “plasma concentration—time profile”, “Cₘₐₓ, “AUC”,” Tₘₐₓ, and “elimination half life” have their generally accepted meanings as defined in the FDA Guidance for Industry: Bioavailability and Bioequivalence Studies for Orally Administered Drug Products—General Considerations (issued March 2003).

### EMBODIMENTS

One embodiment of the invention is a stabilized, modified release composition comprising a plurality of drug-containing particles comprising active agent-containing core coated with a first and second coating as described herein, wherein the first coating comprises at least one water-insoluble or enteric polymer. The first coating can be disposed directly over the drug-containing core, coated onto a sealant layer that is disposed over the drug-containing core, coated over the second coating, coated over a sealant layer that is disposed over the second coating, etc.

Another embodiment of the invention is directed to a drug delivery system, preferably providing for once or twice daily delivery, comprised of particle drug population, such as one or more timed, pulsatile-release (TPR) particles optionally further combined with immediate-release (IR) particles. A further embodiment is where the drug delivery system is a multi-particle population that provides for a recovery phase for the h-PCFT mediated methylfolate transporters between the initiation of the L-methylfolate release from different particle populations. Furthermore, it is essential to ensure complete release of the dose from dosage form, irrespective of the local pH, prior to its exiting the proximal small intestine (e.g. duodenum jejunum region of the gastrointestinal tract). L-Methyl-folate-containing particles of the present inventions include methylfolate-layered onto inert cores, and pellets or minitablets/microtablets containing L-methylfolate and at least one pharmaceutically acceptable excipient.

Yet another embodiment of the invention is a pharmaceutical composition comprising:

(a) a population CR/TPR particles, wherein each TPR particle comprises a methylfolate-containing particle (a crystal, bead layered with L-methylfolate and optionally a polymeric binder onto an inert core (sugar sphere or cellulose sphere), pellet or minitablet comprising at least one pharmaceutically acceptable excipient);

(b) a first coating that is disposed over the methylfolate-containing particle, comprising at least one enteric polymer to produce a DR coated methylfolate-containing particle;

(c) a second coating that is disposed over said DR coated methylfolate-containing particle, comprising a water-insoluble polymer in combination with an enteric polymer.

This composition is prepared in accordance with the disclosures of U.S. Pat. No. 6,627,223. This embodiment further optionally comprises a second population of IR particles, wherein each IR particle comprises folic acid salt or pharmaceutically acceptable salt thereof.

In a particular embodiment, the TPR coating comprises ethylcellulose (e.g., Ethocel Premium Standard 10 (EC-10 with a viscosity of 10 cps) as the water-insoluble polymer and hypromellose phthalate (e.g., HP-50 or HP-55, the enteric polymer which starts dissolving in a buffer at pH 5.0, 5.5, or above) as the enteric polymer.

Furthermore, in certain embodiments of the invention, each of the methylfolate-containing particles comprises a core comprising L-methylfolate and is coated with one or more functional polymer coatings that impart the desired extended release properties. In a particular embodiment, the methylfolate-containing core comprises L-methylfolate calcium and at least one pharmaceutically acceptable excipient and coated with one or more functional polymer coatings that impart the desired extended release properties. The first coating disposed directly over the methylfolate-containing particle comprises at least one enteric polymer and the second coating disposed over the first enteric/DR coating layer comprises a lag-time coating comprising an enteric polymer in combination with a water-insoluble polymer. The first and second coatings can be applied in any order. Further, the first coating comprising a delayed release polymer is disposed over a protective seal- or under-coat disposed over the methylfolate-containing particle, followed by the second coating comprising an enteric polymer in combination with a water insoluble polymer. Alternatively, the first coating comprises a combination of enteric and water insoluble polymers applied over the methylfolate-containing particle, followed by a second delayed release coating. Other coatings in addition to the first and second coating can also be applied (e.g., seal coat or an extended release coating) in any order, i.e., prior to, between, or after either of the first and second coatings.

Unless stated otherwise, the amount of the various coatings or layers described herein (the “coating weight”) is expressed as the percentage weight gain of the particles or beads provided by the dried coating, relative to the initial weight of the particles or beads prior to coating. Thus, a 10% coating weight refers to a dried coating that increases the weight of a particle by 10%.

In yet another embodiment, the enteric or lag-time coating polymer may include a plasticizer. The amount of plasticizer required depends upon the plasticizer, the properties of the water-insoluble polymer, and the ultimate desired properties of the coating. Suitable levels of plasticizer range from about 1% to about 20%, from about 3% to about 20%, about 3% to about 5%, about 7% to about 10%, about 12% to about 15%, about 17% to about 20%, or about 1%, about 2%, about 3%, about 4%, about 5%, about 6%, about 7%, about 8%, about 9%, about 10%, about 15%, or about 20% by weight relative to the total weight of the coating, inclusive of all ranges and sub-ranges there between.

In certain embodiments of the invention, the plasticizer may constitute from about 3% to about 30% by weight of the polymer(s) in the controlled-release coating. In still other embodiments, the amount of plasticizer relative to the weight of the polymer(s) in the controlled-release coating is about 3%, about 5%, about 7%, about 10%, about 12%, about 15%, about 17%, about 20%, about 22%, about 25%, about 27%, and about 30%, inclusive of all ranges and sub-ranges there between. The presence of plasticizer, or type(s) and amount(s) of plasticizer(s) can be selected based on the polymer or polymers and nature of the coating system (e.g., aqueous or solvent-based, solution or dispersion-based and the total solids).

In certain embodiments of the invention, the compositions may comprise a combination of IR and DR, IR and SR or IR and TPR coated multiple units, wherein the TPR coating is applied over IR particles, DR or SR coated multiunits such that the total (DR or SR & TPR) coating is applied for a coating weight of about 5% to about 25% by weight, including the ranges of from about 5% to about 20%, and from about 10% to about 15%, inclusive of all ranges and sub-ranges there between while the individual SR, DR or TPR coating has to be at least one percent w/w.

As described herein, in various embodiments the controlled release compositions of the invention comprise a plurality of L-methylfolate calcium-containing particles, coated with a first coating of a DR layer (comprising an enteric polymer) and a second coating of a TPR coating layer (comprising a combination of enteric and water-insoluble polymers).

In yet another embodiment of the invention, the controlled release composition may further comprise a seal coat layer disposed on the L-methylfolate calcium-containing particles, e.g. between the first and second coatings, beneath the first and second coatings, and/or over both of the first and second coatings to prevent (or minimize) static and/or particle attrition during processing and handling.

In one embodiment, the seal coat layer comprises a hydrophilic polymer. Non-limiting examples of suitable hydrophilic polymers include hydrophilic hydroxypropylcellulose (e.g., KLUCEL® LF), hydroxypropyl methylcellulose or hypromellose (e.g., OPADRY® Clear or PHARMACOAT™ 603), OPADRY II, vinylpyrrolidone-vinylacetate copolymer (e.g., KOLLIDON® VA 64 from BASF), and ethylcellulose, e.g. low-viscosity ethylcellulose. The seal coat layer can be applied at a coating weight of about 1% to about 10%, for example about 1%, about 2%, about 3%, about 4%, about 5%, about 6%, about 7%, about 8%, about 9%, or about 10%, inclusive of all ranges and sub-ranges there between.

Still another embodiment according to the invention is directed to the CR composition comprising both IR and TPR particle populations, wherein said composition provide complete release in about 5 hours when dissolution tested using United States Pharmacopoeia (USP) dissolution methodology (Apparatus 2—paddles@ 50 RPM, 0.1N HCl at 37° C. for one hour and in the phosphate buffer at pH 5.8 thereafter).

In a particular embodiment, methylfolate-containing composition is a blend comprising L-methylfolate in combination with one or more pharmaceutically acceptable excipients (e.g., lactose, mannitol, dibasic calcium phosphate, microcrystalline cellulose, sodium starch glycolate (EXPLOTAB®, a disintegrant), at least one dissolution rate controlling hydrophilic polymer. Such a blend may include a suitable lubricant and optionally a binder and can be compressed into controlled-release matrix tablets using a conventional rotary tablet press as described herein.

Non-limiting examples of suitable disintegrants include sodium starch glycolate, crospovidone (cross-linked polyvinylpyrrolidone), carboxymethylcellulose sodium (AC-DI-SOL®), low-substituted hydroxypropylcellulose, corn starch and mixtures thereof.

Non-limiting examples of suitable binders include povidone (polyvinylpyrrolidone), hydroxypropylcellulose, hypromellose (hydroxypropylmethylcellulose (HPMC)), corn starch, pregelatinized starch and mixtures thereof.

In certain embodiments, non-polymeric materials such as non-polymeric waxes and fatty acid esters may be used instead of hydrophilic, water-swellable or hydrophobic polymers.

In another embodiment of the invention, the compositions further comprise a number of pharmaceutically acceptable excipients selected from the group consisting of dibasic calcium phosphate, calcium sulphate, microcrystalline cellulose, lactose, mannitol, polyvinylpyrrolidone, functional or dissolution rate controlling polymers such as ethylcellulose, hydroxypropyl methylcellulose, hydroxypropylcellulose, hydroxyethylcellulose, carbopols, polyethylene oxides, tragcanth gum, alginic acid, carrageenans, alginates, fatty acids, fatty acid esters, sodium starch glycolate, carboxymethyl cellulose sodium, polyvinyl acetate, and acrylate-methacrylic acid copolymers to form robust CR matrix tablets providing target PK profiles to be suitable for a once-daily dosing regimen in depressed patients. The amount of each of these excipients in the composition of CR matrix tablets may vary from about 0.5% to about 95% of the tablet weight.

Another embodiment of the invention is directed to two or more pharmaceutically acceptable excipients blended with drug-containing particles, which can be optionally granulated via the use of a conventional wet or dry granulation process, and compressed into matrix tablets wherein the functional polymers by virtue of their physicochemical properties control the drug release by diffusion, erosion, and/or combination thereof through the swollen matrix. The matrix tablet so produced is optionally further coated with a cosmetic, moisture and/or light barrier film coating. Alternatively, the matrix tablet is optionally further coated with functional polymers to further modulate drug release profiles.

Another embodiment of the invention is directed to a stabilized modified-release (MR) dosage form comprising the active agent, such as L-methylfolate, in up to 50 mg dosage strength having a membrane coating on modified release unit dosage forms. The MR dosage form in certain embodiments of the present invention comprise at least one hydrophilic dissolution rate controlling polymer and at least bioadhesive polymer, and the individual units, polymer matrix based tablets, minitabs and microtabs (small tablets 2-3 mm and <2 mm in diameter, respectively, and drug-containing particles including granules, drug-layered beads, extruded-spheronized pellets that can be coated with one or more functional polymers, and filled into capsules, will have at least one protective stabilizing coating. Furthermore, the unit dosage forms may be filled into lower moisture permeable HPMC capsules or induction-sealed glass or HDPE bottles with oxygen scavengers and 2-in-1 desiccants or cold form or Alu-Alu (aluminum-aluminum) blisters, thereby further improving the stability of the MR dosage forms of the present invention.

Still another embodiment according to the invention is directed to a tablet composition comprising L-methylfolate and one or more pharmaceutically acceptable excipients including functional polymers wherein the functional polymers control drug release under in vitro dissolution testing conditions as well as provide target pharmacokinetic profiles of L-methylfolate calcium having an absorption window (i.e., primarily absorbed in the duodenum jejunum region of the gastrointestinal tract) via the saturable h-PCFT mediated methylfolate transport to be suitable for a once-daily dosing regimen in patients with MDDs and/or diagnosed with dysthymia, schizophrenia, or degenerative dementia of the Alzheimer type.

Still another embodiment according to the invention is directed to the CR matrix tablet composition, wherein said composition provides complete release in about 5 hours when dissolution tested using United States Pharmacopoeia (USP) dissolution methodology (Apparatus 2—paddles@ 50 RPM, 0.1N HCl at 37° C. for three hours and in the phosphate buffer at pH 5.8 thereafter).

A stabilized composition according to the invention would be useful for efficacious management of a MDD and/or treating patients diagnosed with dysthymia, schizophrenia, degenerative dementia of the Alzheimer type, endothelial dysfunction associated with diabetic peripheral neuropathy. L-methylfolate may be prescribed for up to 12 weeks. In view of non-linear absorption, which is restricted to proximal small intestine, short plasma elimination half-life, and saturable h-PCFT mediated methylfolate transporter, the present invention may be directed to a once- or twice-daily delivery system composition providing exposure of L-methylfolate that is equivalent to or higher than that achievable from IR tablets of equivalent dose strength.

A composition according to the invention, relative to L-methylfolate, is designed to address several formulation challenges. First, L-methylfolate has a short plasma elimination half life of about 3 hours and is prone to hydrolytic and oxidative degradation during processing and storage. Second, absorption of L-methylfolate occurs principally in the proximal small intestine, i.e., duodenum and upper jejunum region, is non-linear via saturable (at 20 mg or above) h-PCFT mediated methylfolate transporters. Thus, a further embodiment of the present invention is a stabilized dosage form as a MR capsule formulation containing two populations of DIFFUCAPS® beads that release L-methylfolate in an IR-like profile with a peak separation of about 0.5-3 hours under in vitro dissolution conditions such that complete drug release is achieved from the dosage form prior to its exiting from proximal small intestine, i.e., the duodenum jejunum region.

It is an objective of the present invention to produce MR L-methylfolate once-daily formulations that would exhibit a sustained plasma profile that is about equivalent to or higher than (enhanced to) that achievable with an equivalent strength immediate-release (IR) dosage form.

Another embodiment of the invention is to provide a method of producing stabilized matrix tablet formulation comprising at least one dissolution rate-controlling matrix material that would exhibit a sustained plasma profile that is about equivalent to or higher than that achievable with an equivalent strength immediate-release (IR) dosage form. The dissolution rate-controlling matrix material is selected from at least one fatty acid, fatty acid ester, water-insoluble, water-swellable, gelling and eroding polymer, and at least one bioadhesive polymer,

MR dosage forms with enhanced (higher) in vivo bioexposure would further enhance the efficacy of L-methylfolate as monotherapy of MDDs and hence the compliance as well as quality of life of patients with MDD and/or schizophrenia.

A folic acid derivative for use in the stabilized dosage form of the present invention, is selected from the group consisting of tetrahydrofolic acid, dihydrofolic acid, 5-formyltetrahydrofolic acid, 10-formyltetrahydrofolic acid, 5,10-methylenetetrahydrofolic acid, 5,10-methenyltetrahydrofolic acid, 5-formiminotetrahydrofolic acid and their polyglutamate derivatives, S-adenosylmethionine salt, 5-methyltetrahydrofolic acid, and 5-formyltetrahydrofolic acid, D-glucosamine folate, D-galactosamine folate, D-glucosamine (6R,S)-tetrahydrofolate, D-glucosamine (6S)-tetrahydrofolate, D-galactosamine (6R,S)-tetrahydrofolate, D-glucosamine 5-methyl-(6S)-tetrahydrofolate (as disclosed in U.S. Pat. No. 5,817,659; U.S. Pat. No. 6,441,168; U.S. Pat. No. 6,995,158; U.S. Pat. No. 6,093,703; U.S. Pat. No. 7,947,662, and U.S. Pat. No. 8,258,115. Also included is a pharmaceutically effective salt of the folic acid derivative. A more desired folic acid derivative salt is L-methylfolate calcium described in U.S. Pat. No. 6,011,040, U.S. Pat. No. 6,271,374, U.S. Pat. No. 6,441,168, and U.S. Pat. No. 6,995,158.

Yet another embodiment of the present invention also provides for taste-masking of a component of the composition in the form of an orally disintegrating tablet which rapidly disintegrates upon contact with saliva in the oral cavity forming a smooth, easy-to-swallow suspension containing functional polymer-coated methylfolate-containing multiparticulates. Such tablets meet the FDA recommended disintegration time specification of not more than (NMT) 30 seconds when tested for disintegration time by the USP method <701>.

Another embodiment according to the invention is a pharmaceutical composition in a compressed tablet form comprising multiparticulates, wherein each particle comprises L-methylfolate or at least one pharmaceutically acceptable excipient and wherein said tablet composition provides for a target in vitro release profile as well as a target PK profile of L-methylfolate predominantly absorbed from the duodenum jejunum region of the gastrointestinal tract via the saturable h-PCFT mediated methylfolate transporters, to be suitable for a once- or twice-daily dosing regimen.

Examples of water-soluble polymers include (but are not limited to) methylcellulose, hydroxypropyl cellulose, hydroxypropyl methylcellulose, polyethylene glycol, and polyvinyl pyrrolidone.

Still another embodiment according to the invention is directed to a method of treating a patient subject to, comprising administering a therapeutic effective amount of the composition of the invention comprising IR and TPR L-methylfolate calcium particle populations to the patient in need thereof. The TPR particle population comprises a coating of an enteric polymer and a plasticizer followed by a lag-time coating comprising an enteric polymer in combination with a water insoluble polymer and a plasticizer. Non-limiting examples of suitable enteric polymers include anionic polymers. Further non-limiting examples of enteric polymers include hydroxypropyl methylcellulose phthalate, cellulose acetate phthalate, hydroxypropyl methylcellulose acetate succinate, polyvinyl acetate phthalate, pH-sensitive methacrylic acid-methylmethacrylate copolymers that are sold under the trademark Eudragit® (L100, S100, L30D, FS30D) manufactured by Rohm Pharma, shellac, and mixtures thereof. These enteric polymers may be used as a dry powder or an aqueous dispersion. Some commercially available materials that may be used are methacrylic acid copolymers sold under the trademark Eudragit® (L100, S100, L30D, FS30D) manufactured by Rohm Pharma, Cellacefate® (cellulose acetate phthalate) from Eastman Chemical Co., Aquateric® (cellulose acetate phthalate aqueous dispersion) from FMC Corp., and Aqoat® (hydroxypropyl methylcellulose acetate succinate aqueous dispersion) from Shin Etsu K.K. Non-limiting examples of water-insoluble polymers include ethylcellulose, cellulose acetate, cellulose acetate butyrate, polyvinyl acetate, neutral methacrylic acid-methylmethacrylate copolymers, and mixtures thereof. In one embodiment, the water-insoluble polymer is ethylcellulose. In another embodiment, the water-insoluble polymer comprises ethylcellulose with a mean viscosity of 10 cps in a 5% solution in 80/20 toluene/alcohol measured at 25° C. on an Ubbelohde viscometer. Non-limiting examples of suitable plasticizers include glycerol and esters thereof (e.g., monoacetylated glycerides, acetylated mono- or diglycerides (e.g., Myvacet® 9-45)), glyceryl monostearate, glyceryl triacetate, glyceryl tributyrate, phthalates (e.g., dibutyl phthalate, diethyl phthalate, dimethylphthalate, dioctylphthalate), citrates (e.g., acetylcitric acid tributyl ester, acetylcitric acid triethyl ester, tributyl citrate, acetyltributyl citrate, triethyl citrate), glyceroltributyrate; sebacates (e.g., diethyl sebacate, dibutyl sebacate), adipates, azelates, benzoates, chlorobutanol, polyethylene glycols, vegetable oils, fumarates, (e.g., diethyl fumarate), malates, (e.g., diethyl malate), oxalates (e.g., diethyl oxalate), succinates (e.g., dibutyl succinate), butyrates, cetyl alcohol esters, malonates (e.g., diethyl malonate), castor oil, and mixtures thereof.

Still another embodiment according to the invention is directed to a method of treating a patient subject to, comprising administering a therapeutic effective amount of the composition of the invention comprising IR and TPR L-methylfolate calcium particle populations to the patient in need thereof. The TPR particle population comprises an enteric polymers include cellulose acetate phthalate, hydroxypropyl methylcellulose phthalate, hydroxypropyl methylcellulose acetate succinate, polyvinyl acetate phthalate, pH-sensitive methacrylic acid/methylmethacrylate copolymers (e.g., EUDRAGIT® L, S and FS polymers), shellac, and mixtures thereof. Some commercially available materials that may be used are methacrylic acid copolymers sold under the trademark EUDRAGIT (L100, S100, L30D) manufactured by Rohm Pharma, CELLACEFATE (cellulose acetate phthalate) from Eastman Chemical Co., AQUATERIC® (cellulose acetate phthalate aqueous dispersion) from FMC Corp., and AQOAT (hydroxypropyl methylcellulose acetate succinate aqueous dispersion) from Shin Etsu® K.K.

Still another embodiment according to the invention is directed to a method of treating a patient subject to, comprising administering a therapeutically effective amount of the composition of the invention comprising stabilized L-methylfolate calcium particles dispersed in a matrix comprising one or more pharmaceutically acceptable excipients including at least one hydrophilic swelling/gelling polymer such as hydroxypropyl cellulose, hypromellose (hydroxypropyl methyl cellulose such as METALOSE 90SH) and at least one bioadhesive polymer such as CARBOPOL 971P or G-71 polymer, polyethylene oxide, POLYOX, fillers such as spray-dried mannitol, lactose, dicalcium phosphate dihydrate, calcium sulfate, silicified microcrystalline cellulose (PROSOLV SMCC 90 or PROSOLV SMCC 90HD), and coated with a stabilizing coating disposed over the tablet core or minitablet cores.

A core thus coated with a drug layer, and lacking extended release coatings has immediate release properties, and can be referred to as an “IR bead” or a “rapid release bead”. The drug can be deposited on core by any suitable method known in the art. For example, the drug can be deposited from a solution or suspension containing a polymeric binder and micronized methylfolate directly onto the inert sugar sphere or cellulose sphere in a fluid-bed coater.

### EXAMPLES

The invention is described in greater detail in the sections below. The following examples are used to illustrate the invention. It should be understood that the examples and embodiments described herein are for illustrative purposes only and that various modifications or changes in light thereof will be suggested to persons skilled in the art and are to be included within the spirit and purview of this application.

### Example 1

1.A L-Methylfolate MR Tablets:

Micronized L-methylfolate calcium (153 g), hypromellose (METALOSE 90SH; 175 g), and crosslinked polyacrylic acid (CARBOPOL 971P; 37.5 g) are blended in a V-blender for 5 min at 26 RPM, hand screened through #40 mesh sieve to deagglomerate, and further blended with sieved (through a 35 mesh screen) citric acid anhydrous (75 g), direct spray-dried mannitol (1934 g), and silicified microcrystalline cellulose (PROSOLV SMCC 90HD; 100 g) for 10 minutes, sieved through 18 mesh screen, and further blended for 2 minutes after adding magnesium stearate (25 g) to produce a homogeneously blended mixture for compression. 50 mg L-methylfolate MR tablets weighing 1 g, hardness of about 18 kP, and 14.21 mm in diameter are produced on the Betapress using 15 mm standard concave round tooling. These 50 mg L-methylfolate MR tablets (2500 g) are provided with a stabilizing protective film coating with OPADRY II Blue (100 g at 15% solids), followed by a coating with carnauba wax (0.25 g) in a pan coater equipped with a 15″ pan and a single gun.

1.B Methylfolate MR Tablets:

MR tablet mix is first prepared by blending micronized L-methylfolate calcium (73.8 parts) and silicified microcrystalline cellulose (PROSOLV SMCC 90; 113.7 parts) in a V-blender for 10 minutes and sieved through 35 mesh screen. The sieved material is blended with silicified microcrystalline cellulose (PROSOLV SMCC 90; 113.7 parts), dibasic calcium phosphate dehydrate (526.1 parts), hypromellose phthalate (HP-50; 36.6 parts), polyethylene oxide (POLYOX (POLYOX WSR 301; 45.5 parts) magnesium oxide (36.3 parts), and sodium ascorbate (36.1 parts) and blended for 10 minutes. Magnesium stearate (18.2 parts) that is sieved through a 35 mesh screen is added to the blend and further blended for 2 minutes producing a homogenous blend for compression. Betapress, equipped with 15 mm standard concave round tooling is used to compress MR tablets weighing 1 g as described above. The 50 mg L-methylfolate calcium MR tablets (2500 g) are provided with a stabilizing protective film coating with OPADRY II Blue, followed by a coating with carnauba wax (0.5 g) in a pan coater.

1.0 Methylfolate MR Tablets:

MR tablets are first prepared by blending micronized L-methylfolate calcium (61 parts), crosslinked polyacrylic acid (CARBOPOL 71G; 120 parts), silicified microcrystalline cellulose (SMCC 90; 180 parts) and silicified microcrystalline cellulose (PROSOLV SMCC 90HD; 180 parts) in a V-blender for 10 minutes and sieving through 35 mesh screen. The sieved material is blended with dibasic calcium phosphate dehydrate (419 parts) and sodium ascorbate (30 parts) and blended for 10 minutes. Magnesium stearate (10 parts) that is sieved through a 40 mesh screen is added to the blend and further blended for 2 minutes producing a homogenous blend for compression. 50 mg L-methylfolate MR tablets weighing 1 g are produced on the Betapress using 15 mm standard concave round tooling. These 50 mg L-methylfolate calcium MR tablets are provided with a stabilizing protective film coating with OPADRY II Blue (100 g at 15% solids), followed by a coating with carnauba wax.

1.D Methylfolate MR Minitablets:

MR minitablets are first prepared by blending micronized L-methylfolate calcium (6.5 parts), hypromellose (K400LV; 3.5 parts), and crosslinked polyacrylic acid (CARBOPOL 71G; 12 parts) and in a V-blender for 10 minutes and sieved through 35 mesh screen. The sieved material is blended with silicified microcrystalline cellulose (SMCC 90; 18 parts), silicified microcrystalline cellulose (SMCC 90HD; 18 parts), dibasic calcium phosphate dehydrate (38 parts) and sodium ascorbate (3 parts) and blended for 10 minutes. Magnesium stearate (1 part) that is sieved through a 35 mesh screen is added to the blend and further blended for 2 minutes producing a homogenous blend for compression. A rotary tablet press, Betapress, equipped with a minitablet tool set (8, each 2 mm in diameter) is set up with the following compression parameters—fill depth: 3 mm; thickness: ˜2 mm; main compression: 2.0 tons; hardness: 2.3 kP; weight ˜8 mg. These L-methylfolate calcium MR minitablets (2000 g) are provided with a stabilizing protective film coating with OPADRY II Blue (100 g at 15% solids), followed by a coating with carnauba wax (0.5 g) in Glatt GPCG 3.

### Example 2

2.A 25 mg IR L-Methylfolate Tablets:

A 0.25 cu-ft V-blender with (1) silicified microcrystalline cellulose (SMCC 90HD; 21.0 parts), (2) silicified microcrystalline cellulose (SMCC 90; 21.0 parts), (3) micronized L-methylfolate calcium (16.6 parts), (4) dibasic calcium phosphate dihydrate (32.4 parts), (5) sodium starch glycolate (EXPLOTAB; 5 parts), and (6) citric acid anhydrous (3 parts), and blending for 5 minutes. The blended material is passed through a #20 mesh screen. The blender is charged with the screened material, blended for 10 minutes, and magnesium stearate (1.0 part) that is sieved through a 35 mesh screen is added to the blender and further blended for 2 minutes producing a homogenous blend for compression (batch size: 1000 g). The blend is discharged into a property labeled, tared, double polyethylene-lined container.

A rotary tablet press is set up with the following parameters: Fill depth: 8 mm; Pre-compression force setting: 6 mm; Main compression force setting: 4.1 mm; No. of tooling: δ 0.63 mm round concave tooling without embossing. The press is started and after a few die table/turret rotations, tablets are collected to test them against the parameters: Weight: 185 (176-192) mg; Thickness: FIO (for information only); Hardness: 80 (60-100) N; Friability: NMT 1%. Also, the tablet's appearance is inspected for picking, capping, etc, and parameters are adjusted as needed. Once tablet properties meet a predetermined target, the tablet press is run in the automode and the tableting parameters are recorded on the tableting log. Tablets are collected in a properly labeled container lined with clean, double PE bags. At the beginning, middle and end of the compression process run, 15 g of tablets are removed, 5 tablets for testing for weight, thickness, and hardness, and 6.5 g of tablets for friability and the rest of the samples as a composite sample. All test results are recorded on the In-Process Compression Data Sheet. If any tablet attributes (hardness, weight, etc.) begin to drift, make the necessary adjustments to bring the tablets back into the target outlined. An adequate product level in the press hopper is maintained and tableting is continued until the material in the supply hopper is depleted. The finished tablets are checked by passing them through the metal detector. The headspace above the bulk tablets is purged with nitrogen, and oxygen absorbing packs are placed in direct contact with the bulk material and one desiccant pack is placed between the polyethylene bags. The polyethylene bags are closed with ties and the lids on the containers are secured and moved to storage.

2.B Methylfolate IR Minitablets:

IR minitablets are first prepared by charging a 0.25 cu-ft V-blender with (1) silicified microcrystalline cellulose (SMCC 90HD; 44.5 parts), (2) silicified microcrystalline cellulose (SMCC 90; 15 parts), (3) micronized L-methylfolate calcium (15 parts), (4) dibasic calcium phosphate dihydrate (15 parts), (5) sodium starch glycolate (EXPLOTAB; 5 parts), and (6) citric acid anhydrous (5 parts), and blending for 5 minutes. The blended material is passed through a Comil equipped with a 032R screen at an impeller speed of approximately 2400 rpm. The blender is charged with the screened material and magnesium stearate (0.5 part) that is sieved through a 35 mesh screen is added to the blender and further blended for 2 minutes producing a homogenous blend for compression (batch size: 2 kg).

A rotary tablet press, Manesty Betapress, equipped with a minitablet tool set (16, each 2 mm in diameter) is set up with the following compression parameters—fill depth setting: 4 mm; Pre-compression setting: 4 mm main compression setting: 4.0 mm; Force feeder setting: 1; Weight of 10 minitablets: 80 (75-85) mg; Individual weight: 7.0-9.0; hardness: 20 (10-30) N. After achieving target weight and hardness, the tableting process is continued while taking approximately 1 g of minitablets for determining the weight of 10 units and individual weight, thickness, and hardness values of 5 units. If any tablet attributes begin to drift, necessary adjustments are made and adjusted parameters are recorded on the batch record. Minitablet cores (1100 g) are provided with a stabilizing coating comprising an OPADRY II Blue coating (110 g) dissolved/dispersed in 440 g of USP water in a Glatt GPCG 3 equipped with a 7″ Wurster insert, peristaltic pump and 1.0 mm nozzle tip size for a spray rate of 8 mL/minute, Air distribution plate ‘D’ and 200 mesh product support screen, and dedicated filter bag at the following parameters: Inlet temperature setting—55° C.; Process air volume—70 cfm; Atomization air—2.0 bar; Target product temperature: 37-38° C.

2.0 TPR Minitablets:

The DR membrane coating solution is prepared by adding 93.9 g of water into 1784.5 g of acetone in a stainless steel container while stirring. Hypromellose phthalate, HP-50 (see Table 1 for compositions/batch quantities) is added to the solvent mixture while stirring until dissolved, and triethyl citrate (TEC) is added while stirring for not less than 30 minutes. Minitablet cores from Example 2.B above are first coated with the DR coating solution in Glatt GPCG 3 for a coating weight gain of 13.98% under the following steady-state conditions—bottom air distribution plate: ‘D’ and 200 mesh product screen; atomization air pressure: 1.5 bar; nozzle port size: 1.0 mm; inlet temperature: 37° C.; product temperature: 33-34° C.; flow rate: 4, 8, 12, 18 mL/min; and air flow: 60-40 CFM. The coated minitablets are further coated with a lag-time coating formulation [(EC-10; 11.2 g), HP-50 (11.2 g), and TEC (2.49 g) dissolved in 95/5 acetone/water] to produce TPR minitablets with a weight gain of 1.28% by weight for dissolution testing. Another minitablet prototype having a DR coating at 14% by weight is further coated with a lag-time coating of 1%, 2%, 3% by weight for drug release testing.

TABLE 1
Compositions of TPR minitablets

| | | | |
|---|---|---|---|
| Unit Qnty, mg | g/batch | Unit Qnty, mg | g/batch |

| | | |
|---|---|---|
| Ingredients | Example 2.B (Non-talc) | Example 2.C (with talc) |

DR coating

| L-Methylfolate Minitablets | 8.00 | 1100.0 | | 8.00 | 1100.0 | |
|---|---|---|---|---|---|---|
| Hypromellose | 1.106 | 228⁽¹⁾ | (152)⁽²⁾ | 0.61 | 268⁽¹⁾ | (84.1)⁽²⁾ |
| Phthalate NF (HPMCP-50) | | | | | | |
| Triethyl Citrate NF | 0.195 | 40.5⁽¹⁾ | (27)⁽²⁾ | 0.11 | 48.5⁽¹⁾ | (15.2)⁽²⁾ |
| Talc USP | | | | 0.58 | 254⁽¹⁾ | (79.7)⁽²⁾ |

TPR or Lag-time coating

| | | | | | | |
|---|---|---|---|---|---|---|
| Ethyl cellulose | 0.054 | 11.20⁽¹⁾ | (7.47)⁽²⁾ | 0.03 | 44⁽¹⁾ | (4.1)⁽²⁾ |
| Hypromellose Phthalate NF | 0.054 | 11.20⁽¹⁾ | (7.47)⁽²⁾ | 0.03 | 44⁽¹⁾ | (4.1)⁽²⁾ |
| (HPMCP-50) | | | | | | |
| Triethyl Citrate NF | 0.012 | 2.49⁽¹⁾ | (1.66)⁽²⁾ | 0.01 | 14⁽¹⁾ | (1.4)⁽²⁾ |
| Talc USP | | | | 0.05 | 73.5⁽¹⁾ | (7.0)⁽²⁾ |

| | | | | |
|---|---|---|---|---|
| Total | 9.42 | 1295.6 | 9.42 | 1295.6 |

⁽¹⁾An excess coating suspension dispensed to account for process losses. The acetone:water ratio is 95:5 and solids content of the coating suspension is 16%.
⁽²⁾Theoretical quantity required.

2.D Further DR/TPR Coatings Containing Talc:

In order to examine the effect of talc in DR and TPR coating formulations on the in vitro release of L-methylfolate calcium from TPR minitablets, talc is included in the DR, as well as the lag-time (TPR) coating formulations, at a weight ratio of total (polymer+plasticizer) to talc of about 55:45. DR coating trials are performed for a weight gain of 13.8%, 26.5%, or up to 30% by weight of the DR minitablets. DR minitablets having 30% coating by weight of the total DR minitablets are coated with a lag-time coating containing talc for a weight gain of up to 10% by weight of the TPR minitablets. DR minitablets at 13.8% or 26.5% coating are further coated with a lag-time coating of 1.3% or 1% by weight.

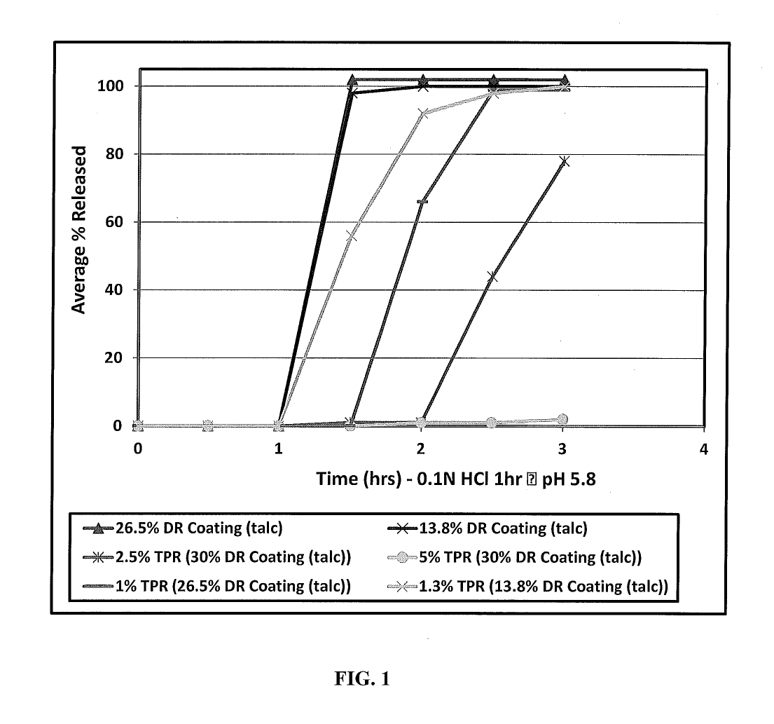

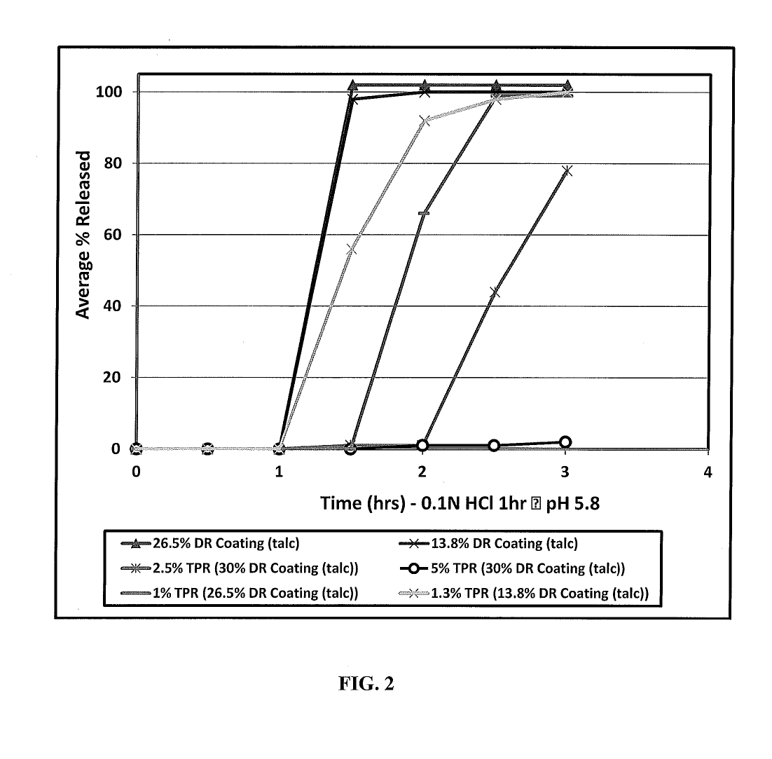

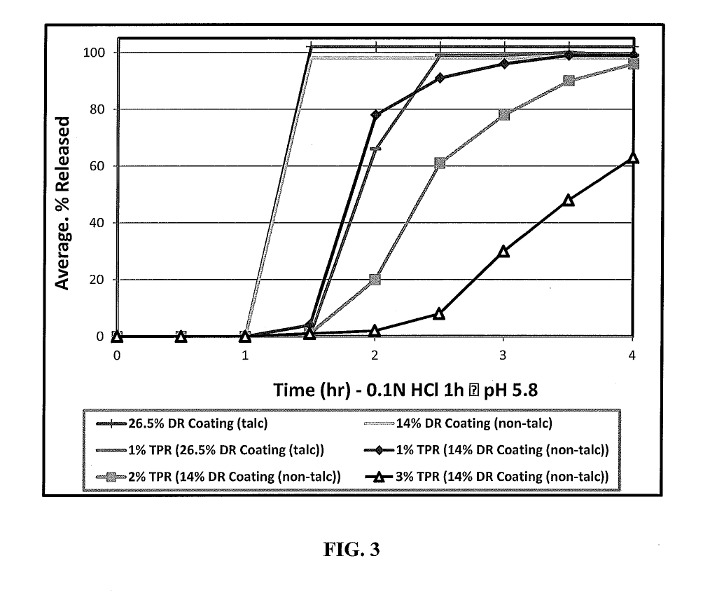

The data in FIG. 1 show that the (talc-containing) DR-coated minitablets exhibit negligible drug release in the acidic buffer for 1 hour. Upon exposure to pH 5.8 dissolution media, L-methylfolate calcium is rapidly released from the TPR minitablets coated at 13.8% DR coating and 1.3% TPR coating by weight, releasing 76% of the drug within 30 minutes and 92% at 1 hour. However, the results from the TPR minitablets having a 30% DR coating, show that the increasing TPR coating level results in increasing lag time, as demonstrated in FIG. 2. Following the lag-time of 2 hrs, the TPR minitablets at 2.5% lag-time coating release 41% and 80% of the drug, respectively, at 2.5- and 3-hour time points. At a lag-time coating of 5% or higher, the lag time is longer than 4 hours. Even the TPR minitablets at 26.5% DR and 1% and TPR coating release 66% and 98% of the drug, respectively, at 2.0- and 2.5-hour time points. From FIG. 3 it is evident that the DR minitablets having a 26.5% talc-containing DR membrane coating provides a drug release profile similar to that achieved from DR minitablets coated with only 14% non-talc DR membrane coating, thereby suggesting a limited impact of talc in modulating L-methylfolate release. However, from a comparison of the L-methylfolate release profiles from TPR [2.5% TPR coating over 30% DR membrane coating (talc)] and TPR [3% TPR coating over 14% DR membrane coating (non-talc)] minitablets shown in FIGS. 2 and 3, respectively, it appears the use of talc in the DR and TPR membrane could result in sharper release profiles following the lag-time.

2.E Minitablets MR Capsules:

One 25 mg IR tablet equivalent to 25 mg L-methylfolate (relative to free acid) from Example 2.A and required amount of TPR minitablets equivalent to 25 mg L-methylfolate (relative free acid) from Example 2.D (1.3% lag-time coating layer disposed over 13.8% DR coated minitablet population) are filled into HPMC capsules for analytical testing.

### Example 3

3.A Methylfolate MR Tablets:

Micronized L-methylfolate calcium (see Table 2 for compositions), approximately 3/4 of hypromellose (METOLOSE 90SH), and CARBOPOL 971P are blended in a 0.5 cu-ft V-blender for 5 min at 26 RPM, screened to deagglomerate and rinsed with 1/4 of hypromellose, and further blended with sieved (through a 35 mesh screen) citric acid anhydrous, spray-dried mannitol, and silicified microcrystalline cellulose for 10 minutes, sieved through 18 mesh screen, and further blended for 2 minutes after adding magnesium stearate to produce homogeneously blended compression mix. Content uniformity of the blend is confirmed by taking samples from equidistance-spaced locations in the powder bed using a 5 compartment sample thief.

TABLE 2
Compositions of MR Tablets

| | 20 mg MR Tablets | | 50 mg MR Tablets | |
|---|---|---|---|---|
| Ingredient | mg/tablet | g/batch | mg/tablet | g/batch |
| L-Methylfolate calcium | 24.7 | 185.2 | 61.34 | 460 |
| Hypromellose (Metalose 90SH) | 72.9 | 546.4 | 70.0 | 525 |
| Silicified MCC (SMCC 90HD) | 40.0 | 300.0 | 40.0 | 300 |
| Mannitol | 815.4 | 6115.5 | 773.7 | 5803 |
| Carbopol 971P | 7.0 | 52.9 | 15.0 | 112.1 |
| Citric acid anhydrous | 30.0 | 225.0 | 30.0 | 225 |
| Magnesium stearate | 10.0 | 75.0 | 10.0 | 75 |
| Total tablet core | 1000.0 | 7500.0 | 1000.0 | 7500 |
| Opadry II Blue | 30.9 | 370.8 ⁽¹⁾ (231.8) ⁽²⁾ | 0.31 | 353.4 ⁽¹⁾ (232) ⁽²⁾ |
| Carnauba wax | 0.1 | 0.75 | 0.1 | 0.75 |
| Total tablet weight | 1031 | 7732.5 | 1031 | 7813.0 |

⁽¹⁾ An excess coating solution dispensed to account for process losses. The solids content of the coating solution is 15%.
⁽²⁾ Theoretical quantity required.

50 mg L-methylfolate MR tablets are compressed on the Betapress under the conditions shown in the table below. During the compression run, 15 tablet samples are taken—5 tablets for individual measurement of tablet weight, thickness, 5 tablets for content uniformity testing, and hardness and 5 more as a composite sample for analytical testing. 10 tablets are sampled at the beginning, middle, and end of run for friability testing.

| Tooling | 19 | mm Oval |
|---|---|---|
| Number of stations | 8 | |
| Overload pressure | 2.5 | tons |
| Fill depth | 10 | mm |
| Thickness setting | 4.1 | |
| Force feeder setting | 5 | |
| Tablet fill weight (mg) | 1000 | (970-1030) |
| Tablet thickness (mm) | 6.8 | |
| Tablet hardness (kP) | 17 | (16-18) |

These 50 mg L-methylfolate MR tablets (6500 g) have been coated with a stabilizing film coating at 3% weight gain comprising an aqueous solution of OPADRY II Blue (232 g at 15% solids, followed by waxing with carnauba wax (0.75 g) in a pan coater equipped with a 24″ pan and two guns at the following conditions—inlet temperature: 60° C. (59-65° C.); exhaust temperature: 46° C. (43-49° C.); air volume: 168 CFM (167-172); atomizing air pressure: 16.5 psi; pan speed: 10 rpm; and spray rate: 10 g/min per gun. After completion of the coating, carnauba wax is added into the product bowl prior to cooling down. The MR tablets are discharged into light protected containers. The film coated MR tablets show an average hardness of 20.3 kP and a friability of 0.12%. The MR tablets are packaged in 100 cc nitrogen purged, induction-sealed HDPE bottles (50′ count) with a cotton coil, desiccant pack, and closure, and then stability tested at 25° C./60% RH. The MR tablets show acceptable physical and chemical stability profiles at 6 month time point.

Mechanism of L-Methylfolate Release from MR Tablets:

Without being bound by the exact mechanism of L-methylfolate release and/or absorption, large matrix tablets comprising swelling, mucoadhesive and dissolution-rate controlling polymers slowly release L-methylfolate during in vitro dissolution testing and are expected to release L-methylfolate for absorption in duodenum and upper jejunum following oral administration in healthy volunteers and/or patients diagnosed with MDDs by:
initial hydration of the matrix polymers causing significant increase in volume of the matrix tablet
diffusion of solubilized L-methylfolate through the hydrated matrix layer over time, erosion of the matrix polymers and tablet disintegration and/or exiting the stomach.

3.B 20 mg Methylfolate MR Tablets:

20 mg MR tablets having the composition listed in Table 2 are prepared and provided with a stabilizing film coating following the procedures disclosed in Example 3.A.

3.C 50 mg L-Methylfolate IR Tablets:

A 0.25 ft³ V-blender is charged with (1) approximately one-half of dibasic calcium phosphate dihydrate (see Table 3 for the composition and batch quantities), (2) approximately one-half of micronized L-methylfolate calcium, (3) remaining dibasic calcium phosphate dihydrate, (4) remaining L-methylfolate and blended for 10 min at 26 rpm. The silicified microcrystalline cellulose (SMCC 90), the above pre-blend, and the silicified microcrystalline cellulose (SMCC 90HD) are passed through a Comil equipped with a 062R screen (spacer 0.325″) at 1300 rpm to deagglomerate. A 0.5 ft³ V-blender is charged with the Comilled material and blended for 10 minutes to achieve a homogenized blend. The blended material is again passed through the Comil at 1300 rpm. The 0.5 ft³ V-blender is charged again with the Comilled material and blended for 5 minutes. Magnesium stearate is hand screened through a 35 mesh sieve, added into the blender, and further blended for 2 minutes to produce homogeneously blended compression mix.

TABLE 3
Compositions of IR Tablets

| | 19.5 mg IR Tablets | | 50 mg IR Tablets | |
|---|---|---|---|---|
| Ingredient | mg/tablet | g per batch | mg/tablet | g per batch |
| L-methylfolate calcium | 24.07 | 240.7 | 61.7 | 347.3 |
| Silicified MCC, SMCC 90 | 335.0 | 3350 | 306.6 | 1724.9 |
| Dibasic calcium phosphate | 335.9 | 3359.3 | 644.8 | 3627.0 |
| dehydrate | | | | |
| Magnesium stearate | 5.0 | 50 | 13.3 | 75.0 |
| Total tablet core | 700.0 | 7000.0 | 1333.0 | 7499.5 |
| Opadry II Blue | 34.5 | 392 ⁽¹⁾ (245) ⁽²⁾ | 55.3 | (497.5) ⁽¹⁾ (3109 ⁽²⁾ |
| Carnauba wax | 0.07 | 0.7 | 0.1 | 0.75 |
| Total tablet weight | 724.57 | 7245.7 | 1388.4 | 7811.1 |

⁽¹⁾ An excess coating solution dispensed to account for process losses. The solids content of the coating solution is 15%.
⁽²⁾ Theoretical quantity required

50 mg L-methylfolate MR tablets are compressed on the Betapress under the conditions shown in Table 4 below. During the compression run, 15 tablet samples are taken—5 tablets for individual measurement of tablet weight, thickness, 5 tablets for content uniformity testing, and hardness and 5 more as a composite sample for analytical testing. 10 tablets are sampled at the beginning, middle, and end of run for friability testing.

TABLE 4
Process parameters for IR Tablets

| Tablets | 50 mg IR Tablets | | 19.6 mg IR Tablets | |
|---|---|---|---|---|
| Tooling and # stations | 19 × 8 | | 18 mm × 8 Oval | |
| | | | shaped tooling | |
| Turret speed | 25 | rpm | 25 | rpm |
| Fill depth setting | 12.5 | mm | 8 | mm |
| Pre-compression force | 6 | mm | 4 | mm |
| setting | | | | |
| Main compression | 5.5 | mm | 53.2 | mm |
| force setting | | | | |
| Force feeder setting | 3 | | 3 | |
| Tablet fill weight (mg) | 1333 | (1266-1400) | 700 | (665-735) |
| Tablet thickness (mm) | 8.1 | | 5.6 | |
| Tablet hardness (N) | 270 | (220-320) | 150 | (120-200) |

| | | |
|---|---|---|
| Friability | NMT 1% | NMT 1% |
| Appearance | No defects | No defects |

These 50 mg L-methylfolate IR tablets are coated with a stabilizing film coating at 3.98% weight gain comprising an aqueous solution of OPADRY II Blue (232 g at 15% solids, followed by waxing with carnauba wax (0.75 g) in a pan coater equipped with a 24″ pan and two guns at the following conditions—inlet temperature: 60° C. (55-65° C.); exhaust temperature: 46° C. (43-49° C.); air volume: 168 CFM (167-172); atomizing air pressure: 16.5 psi; pan speed: 15 rpm; Pump setting: 24 mL/minute. After reaching a 3.98% coating weight gain, carnauba wax is added into the product bowl prior to cooling down. The MR tablets are discharged into light protected containers. The film coated MR tablets show an average hardness of 15-20 kP and a friability of less than 0.5%. The MR tablets are packaged in 100 cc nitrogen purged, induction-sealed HDPE bottles (50′ count) with a cotton coil, one desiccant pack, and closure, and then stability tested at 25° C./60% RH. The MR tablets show acceptable physical and chemical stability profiles at 3 month time point.

3.D 19.5 mg Methylfolate IR Tablets:

A 0.25 ft³ V-blender is charged with (1) approximately half of hypromellose (METOLOSE 90SH), (2) approximately half of micronized L-methylfolate calcium, (3) remaining half of L-methylfolate calcium, and (4) approximately one-third of dibasic calcium phosphate dihydrate (see Table 3 for compositions) and blended for 5 min at 26 rpm. The remaining half of hypromellose, the pre-blend, and remaining dibasic calcium phosphate dihydrate are sequentially passed through a Comil equipped with a 062R screen (spacer 0.325″) at 1300 rpm to deagglomerate. The Comilled material is blended in the 0.5 ft³ V blender for 15 minutes. Magnesium stearate is hand screened through a 35 mesh sieve, added into the blender, and further blended for 2 minutes to produce homogeneously blended compression mix. The IR tablets are compressed into tablets weighing 700 mg and provided with a stabilizing film coating as disclosed for the 50 mg IR tablets above.

### Example 4

4.A CTM Supplies:

50 mg IR tablets having a composition identical to that of Example 3.C, 50 mg MR tablets having a composition identical to that of Example 3.A, 20 mg MR tablets having a composition identical to that of Example 3.B have been manufactured under cGMP conditions. 20 and 50 mg MR Capsules containing IR and TPR minitablets, each equivalent to 10 or 25 mg L-methylfolic acid, wherein TPR minitablets composition identical to that of Example 2.D are manufactured. The CTM supplies aren release tested using qualified analytical methods to support a Phase I PK/food effect and single multi-dose studies. IR tablets, MR tablets and MR Capsules are packaged in 100 cc nitrogen purged, induction-sealed HDPE bottles (50′ count) with a cotton coil, one oxygen scavenger pack, one desiccant pack, and closure, and stability tested at ICH stability conditions (e.g., 25° C./60% RH, 30° C./65% RH, and 40° C./75% RH).

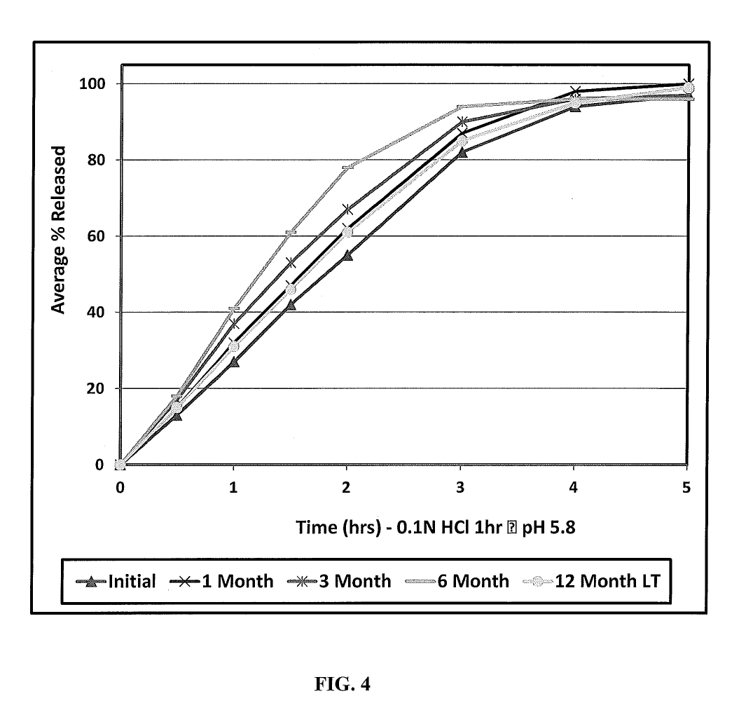

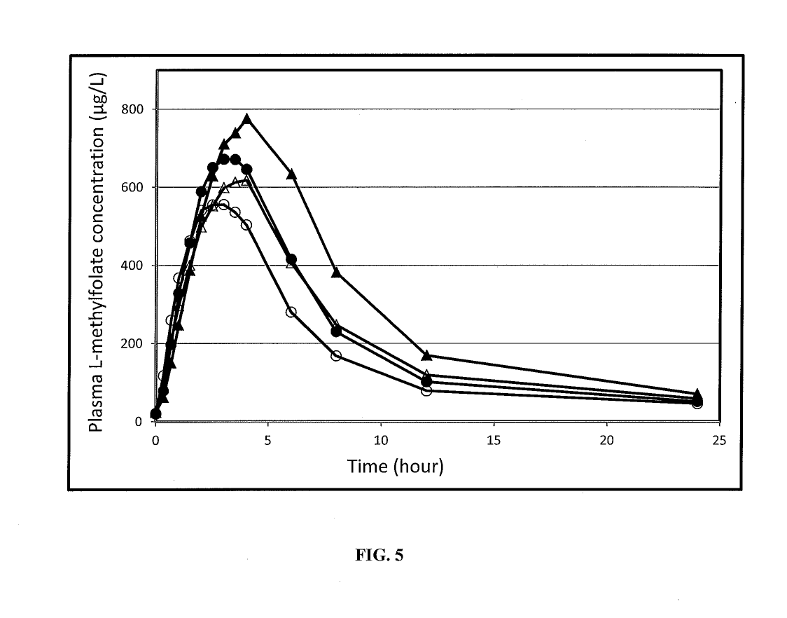

Drug release profiles for the MR tablet batch stability tested at 40° C./75% RH for 6 months are presented in FIG. 4 while their stability data are presented in Tables 5-7. As evident from FIG. 5, the 50 mg MR tablets prototype demonstrates acceptable physical stability under ICH stability conditions. At 40° C./75% RH the dissolution rates increased slightly with time. However, the drug release data from the MR tablets on long term stability at 12 months superimpose on the data from the one-month at 40° C./75% RH MR tablets, thereby confirming the physical stability of the MR tablets. The moisture content of the MR tablets prototypes on stability at ICH conditions remain below 2% by weight. The individual degradant levels of all known or qualified impurities of MR tablets meet the specifications set for the drug substance, which are much tighter than the product specifications (Tables 5 to 7). In contrast, both 19.5 mg (commercial) and 50 mg IR tablets exhibit a moisture level of >4.0% at initial. 50 mg IR Tablets exhibit poor stability at 40° C./75% RH. At 3 and 6-month time points, the IR tablets fail to meet the specifications for some of the qualified impurities set for the drug substance. Also at 6-month time point at 40° C./75% RH, the IR tablets fail to be within the product specification limits for moisture (set at 5%), THFA specified impurity (e.g. 2.5% versus 1.0% for product and 0.5% for drug substance), and total qualified impurities (e.g. 8.4% versus 5% for product and 2.5% for drug substance). Even the 12-month long-term stability for the IR tablets is not encouraging. The MR capsules, 20 mg and 50 mg, and 20 mg MR tablets, and another batch of 50 mg MR tablets show acceptable physical and chemical stability profiles.

TABLE 5
Impurity Profiles for 50 mg IR Tablets (CTM), 50 mg MR Tablets (CTM), and
15 mg IR Tablets (commercial Deplin ®, targeted potency: 19.5 mg overfill)

| | Stability | Stability Timepoint | | | | | | |
|---|---|---|---|---|---|---|---|---|
| Parameter | condition | Initial | 1 Month | 2 Month | 3 Month | 6 Month | 9 Month | 12 Month |
| | | 20 mg MR Tablets (CTM) | | | | | | |
| Moisture (%) | 25° C./60% RH | 0.8 | 0.8 | — | 0.9 | | | |
| Total Impurities(%) | | 1.6 | 1.50 | — | 1.68 | | | |
| Unqualified (%) | | 0.13 RRT0.47; | 0.06 RRT0.49; | <QL RRT0.49; | <QL RRT1.41; | | | |
| | | 0.11 RRT1.27 | 0.09 RRT0.60 | <QL RRT1.28 | <QL RRT1.27 | | | |
| Moisture (%) | 40° C./75% RH | 0.8 | 0.9 | 1.0 | 0.9 | | | |
| Total Impurities (%) | | 1.6 | 1.39 | 1.33 | 1.51 | | | |
| Unqualified (%) | | 0.13 RRT0.47; | <QL RRT0.49; | <QL RRT0.49; | <QL RRT0.51; | | | |
| | | 0.11 RRT1.27 | 0.09 RRT0.59 | <QL RRT1.28 | <QL RRT1.27 | | | |
| | | 50 mg MR Tablets (CTM) | | | | | | |
| Moisture (%) | 25° C./60% RH | 2 | 1.2 | 2.0 | 1.8 | 1.3 | 1.3 | 1.2 |
| Total Impurities(%) | | 1.0 | 1.3 | 1.3 | 1.45 | 1.5 | 1.3 | 1.36 |
| Unqualified | | <QL RRT 1.28; or <QL RRT 1.07; or <QL RRT 0.79; or <QL 0.47 or none | | | | | | |
| Moisture (%) | 40° C./75% RH | 2 | 1.4 | — | 1.7 | 1.4 | | |
| Total Impurities (%) | | 1.0 | 1.3 | — | 1.31 | 2.16 | | |
| Unqualified (%) | | <QL RRT 1.28; or 0.1% RRT 0.79; or 0.06% RRT 0.50; or 0.03% RRT 0.90 or none | | | | | | |
| | | 50 mg IR Tablets, (CTM) | | | | | | |
| Moisture (%) | 25° C./60% RH | 4 | 2.8 | 4.0 | 3.1 | 3.3 | 4.5 | 4.5 |
| Total Impurities (%) | | 1.3 | 1.3 | 1.5 | 1.47 | 1.68 | 2.1 | 2.17 |
| Unqualified (%) | 40° C./75% RH | <QL RRT 1.28; or 0.08% RRT 0.79; or 0.2-0.26% RRT 0.47 or none | | | | | | |
| Moisture (%) | (30° C./65% RH) | 4 | 4.6 | — | 5.1 | 6.7 (3.9) | (4.2) | (4.7) |
| Total Impurities (%) | | 1.3 | 1.15 | — | 2.63 | 8.4* (2.1) | (2.3) | (3.15) |
| Unqualified (%) | <QL RRT 1.28; or 0.24% RRT 0.48; or 0.5% RRT 0.50; 0.27% RRT 1.38; & 0.11% RRT 1.36 | | | | | | | |
| | | Deplin (15 mg Commercial IR tablets; 19.5-mg overfilled) | | | | | | |
| Moisture (%) | 25° C./60% RH | 4.2 | 3.4 | 3.3 | 3.6 | 4.3 | | |
| Total Impurities (%) | | 3.1 | 3.6 | 3.8 | 3.9 | 3.8 | | |
| Unqualified (%) | | 0.7 | 0.6 | 0.7 | 0.6 | 0.4 | | |

*Out of Specification Results: ABGA (0.8%), HOMeTHF (0.31%), MeFOX (1.25%), THFA (2.35%), FA (0.65%), DHFA (0.18%), DiMeTHFA (0.06%), CH2THFA (0.07%), MeTHPA (<QL) observed for IR tablets (CTM); RRT—Relative retention time

TABLE 6
Impurity Profiles for IR Tablets, 50 mg (CTM), MR Tablets, 50 mg (CTM) at 40° C./75% RH

| | | | Individual Impurity levels (%) of IR/MR Tablets (CTM) | | | | | | | |
|---|---|---|---|---|---|---|---|---|---|---|
| | Specifications | | | | | | | | | |
| Specified | NMT | | 50 mg MR Tablets | | | | 50 mg IR Tablets | | | |
| Impurities | API | DP | Initial | 1 Month | 3 Month | 6 Month | Initial | 1 Month | 3 Month | 6 Month |
| ABGA | 0.5% | 1.0% | 0.1% | 0.13% | 0.16% | 0.31% | 0.1% | 0.15% | 0.34% | 0.80% |
| HOMeTHF | 1.0% | 1.0% | 0.2% | 0.09% | ND | 0.08% | 0.40% | ND | 0.13% | 0.31% |
| MeFOX | 1.0% | 2.5% | 0.6% | 0.68% | 0.72% | 0.91% | 0.70% | 0.62% | 0.85% | 1.25% |
| THFA | 0.5% | 1.0% | 0.10% | 0.20% | 0.25% | 0.27% | 0.10% | 0.17% | 0.30% | 2.35% |
| FA | 0.5% | 1.0% | ND | ND | <QL | 0.14% | ND % | ND | 0.13% | 0.65% |
| DHFA | 0.5% | 1.0% | <QL | 0.16% | 0.17% | 0.24% | <QL | 0.15% | 0.14% | 0.18% |
| DiMeTHFA | 0.15% | 1.0% | <QL | <QL | <QL | <QL | <QL | <QL | 0.06% | 0.06% |
| CH2THFA | 0.5% | 1.0% | <QL | 0.06% | <QL | <QL | <QL | <QL | <QL | 0.07% |
| MeTHPA | 0.5% | 1.0% | <QL | <QL | <QL | <QL | <QL | <QL | <QL | <QL |
| Total | 2.5% | 5.0% | 1.0% | 1.3% | 1.31% | 2.16% | 1.3% | 1.15% | 2.63% | 8.43% |
| Impurities | | | | | | | | | | |
| Unqualified, | 0.1% | 1.0% | <QL | <QL | ND | 0.10% RRT | <QL | 0.06% | 0.24% RRT | 0.54% |
| Individual | | | RRT 1.28 | RRT 1.27 | | 0.79; 0.06% | RRT 1.28 | RRT 1.4 | 0.48; 0.27% | RRT 0.50 |
| | | | | | | RRT 0.50; | | | RRT 1.38; | |
| | | | | | | 0.03% RRT 090 | | | 0.11% RRT 1.36 | |
| % Moisture | | 6 | 1.0 | 1.3 | 1.31 | 2.16 | 4.0 | 4.6 | 5.1 | 6.7 |

QL—quantitation limit; ND—not detected RRT—Relative retention time

TABLE 7
Methylfolate Impurity Profiles for Deplin ® (IR Tablets, 19.5 mg)
& Deplin-like IR Tablets, 50 mg (CTM), MR Tablets 50 mg (CTM) at 25° C./60% RH

| | Specifications | | 50 mg MR Tablets | | | 50 mg IR Tablets | | | Deplin (19.5 mg IR tablets) | |
|---|---|---|---|---|---|---|---|---|---|---|
| Specified | NMT | | (CTM) | | | (CTM) | | | (CTM) | |
| Impurities | API | DP | Initial | 6 Month | 12 Month | Initial | 6 Month | 12 Month | Initial | 6 Month |
| ABGA | 0.5% | 1.0% | 0.1% | 0.13% | 0.21% | 0.1% | 0.15% | 0.65% | 0.6% | 0.40% |
| HOMeTHF | 1.0% | 1.0% | 0.2% | 0.11% | 0.19% | 0.4% | 0.16% | 0.44% | 0.5% | 0.4% |
| MeFOX | 1.0% | 2.5% | 0.6% | 0.76% | 0.80% | 0.70% | 0.80% | 0.29% | 1.6% | 1.8% |
| THFA | 0.5% | 1.0% | 0.10% | 0.23% | 0.16% | 0.1% | 0.23% | 0.18% | 0.4% | 0.4% |
| FA | 0.5% | 1.0% | ND | ND | <QL | ND | 0.06% | 0.19% | 0.2% | 0.2% |
| DHFA | 0.5% | 1.0% | <QL | 0.15% | <QL | <QL | 0.19% | <QL | 0.1% | 0.1% |
| DiMeTHFA | 0.15% | 1.0% | <QL | ND | <QL | <QL | <QL | 0.14% | 0.1% | 0.1% |
| CH2THFA | 0.5% | 1.0% | <QL | 0.07% | <QL | <QL | <QL | ND | 0.1% | ND |
| MeTHPA | 0.5% | 1.0% | <QL | <QL | <QL | <QL | <QL | <QL | 0.1% | <QL |
| Total | 2.5% | 5.0% | 1.0% | 1.53% | 1.36% | 1.3% | 1.68% | 3.15% | 3.1% | 3.8% |
| Impurities | | | | | | | | | | |
| Unqualified | 0.1% | 0.2% | <QL | 0.08% | <QL | <QL | 0.08% | 0.14% RRT | 0.1% RRT | 0.1% RRT |
| Individual | | | RRT 1.28 | RRT 0.79 | RRT 1.27 | RRT 1.28 | RRT 0.79 | 0.52; 0.1% | 0.49; 0.1% | 0.55; 0.1% |
| | | | | | | | | RRT 0.96 | RRT 0.55; | RRT 0.71; |
| | | | | | | | | | 0.1% RRT 0.91 | 0.1% RRT 0.79 |
| % Moisture | | 6 | 1.0 | 1.53 | 1.36 | 4.0 | 3.3 | 4.7 | 4.2 | 4.3 |

QL—quantitation limit; ND—not detected RRT—Relative retention time

4.B L-Methylfolate PK and Food Effect Study:

A Phase 1, randomized, parallel group, safety and food effect study comparing the pharmacokinetics (PK) of a single IR tablet, MR tablet, or MR capsule containing 50 mg calcium salt of 6(S)-5-methyltetrahydrofolic acid administered orally in parallel groups each of 20 healthy, adult subjects satisfying all entry (inclusion and exclusion) criteria has been performed. The safety profile of L-methylfolate calcium after oral administration in healthy, adult subjects was also examined by evaluating the frequency and severity of AEs. The first administration was given with half of subjects fasted and half of subjects fed conditions, followed by the second dose under reciprocal feeding conditions after a seven day washout period following the first dose. For all subjects, blood samples for PK analysis were collected at specified time points: immediately before dosing (Time 0) and at 20 minutes, 40 minutes, and 1, 1.5, 2, 3, 4, 6, 8, 12, and 24 hours after dosing. Plasma was prepared, and L-methylfolate plasma concentration was determined by using stabile-isotope dilution LC-ESI-MS/MS (liquid chromatography-electrospray injection tandem mass spectrometry).

Mean concentration-time profiles under fasted and fed states are depicted in FIG. 5. Summarized in Table 8 are the PK values including the % variability and ratio of PK parameters, for L-methylfolate after administration of a single dose of 50 mg IR Tablets, MR tablets, or MR capsules under fasted and fed state. Under fasted and fed conditions, both MR tablets and MR capsules exhibit higher absorption. Statistically significant improvements in absorption in the presence of food are evident in both tablet formulations, especially so in the case of MR Tablets.

TABLE 8
PK parameters for IR MR tablet and MR capsule under fasted and fed conditions

| | | | Cmax | | AUC 0-24 | | AUC 0-∞ |
|---|---|---|---|---|---|---|---|
| PK | Tmax | Cmax ± SD | Ratio | AUC 0-24 ± SD | Ratio | AUC 0-∞ ± SD | Ratio |
| parameters | (hrs) | (μg/L) | MR/IR | (μg/L*hr) | MR/IR | (μg/L*hr) | MR/IR |
| | Oral administration of doses under fasted condition | | | | | | |
| IR tablets | 3.0 | 555.680 ± 164.099 | | 4205.566 ± 1210.545 | | 4573.840 ± 1292.450 | |
| | | (29.5%)* | | (28.0%) | | (28.2%) | |
| MR capsules | 3.5 | 617.737 ± 227.117 | 1.112 | 4945.042 ± 1350.657 | 1.18 | 5411.651 ± 1470.996 | 1.183 |
| | | (36.7%) | | (27.3%) | | (27.1) | |
| MR tablets | 4.0 | 633.600 ± 161.867 | 1.141 | 5192.269 ± 2174.322 | 1.23 | 5711.159 ± 2170.262 | 1.248 |
| | | (25.5%) | | (41.8%) | | (38%) | |
| | Oral administration of doses under fed condition | | | | | | |
| IR tablets | 3.0 | 671.711 ± 211.664 | | 5202.623 ± 1656.546 | | 5593.805 ± 1726.882 | |
| | | (31.5%) | | (31.8) | | (30.9%) | |
| MR capsules | 4.0 | 593.8 ± 118.753 | 0.884 | 5436.355 ± 893.411 | 1.045 | 6138.826 ± 1179.451 | 1.097 |
| | | (19.9%) | | (16.4%) | | (19.2%) | |
| MR tablets | 4.0 | 775.500 ± 102.235 | 1.155 | 6834.177 ± 1649.117 | 1.312 | 7416.356 ± 1082.642 | 1.326 |
| | | (13.2%) | | (24.1%) | | (14.6%) | |

*Percent variability is given within the parentheses.

4.C L-Methylfolate Single and Multiple Dosing Study:

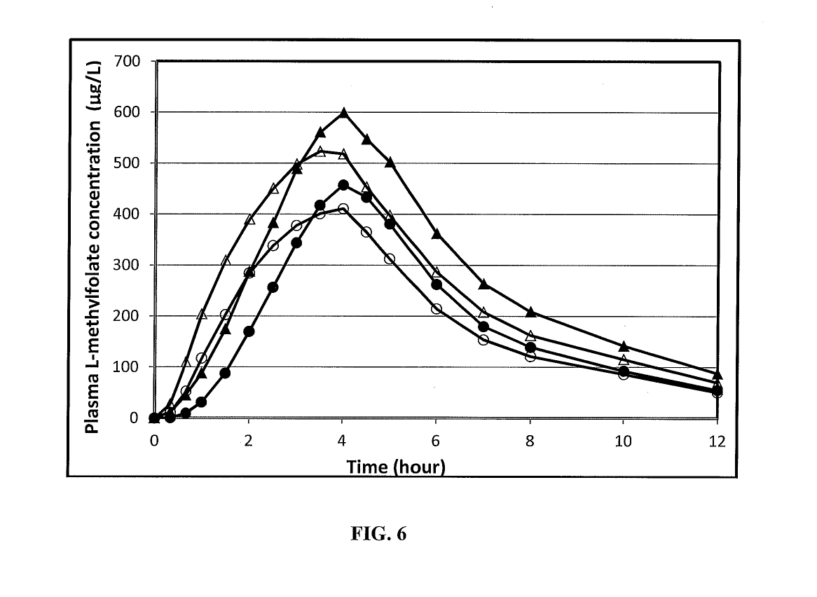

FIG. 6 shows the in vitro dissolution profiles for Deplin® (15 mg (19.5 mg overfilled) and Deplin-like (the same qualitative composition; 50 mg IR tablets), 20 mg and 50 mg MR matrix tablets, and 20 and 50 mg MR capsules that are used for single and multipliple dosing regimens. While the IR tablet prototypes rapidly dissolve, MR capsules took about 2 hrs, and 20 and 50 mg MR matrix tablets took not less than 4 hrs for complete dissolutions. The involves a 6-arm pharmacokinetic and safety study of 6(S)-5-MTHF of calcium comprising a 4-arm, multiple dosing, MR formulations versus a single dose of IR tablets (Deplin® or Deplin®-like (50 mg IR tablets) that are administered orally in parallel dose dependent groups of healthy adult subjects. Subjects for the study are randomized to one of two dose dependent groups, a 50 mg or 20 mg group, and also to a dosing sequence as shown below:
Dosing sequence 1 receives 20 mg MR tablets followed by 20 mg MR capsules and then 19.5 mg IR tablets.
Dosing sequence 2 receives 50 mg MR tablets followed by 50 mg MR capsules and then 50 mg IR tablets.
Within each dose level, subjects are randomized to a dosing sequence to receive either the tablet or capsule MR formulation.
Blood sampling for PK analysis: is collected on the first and last day of each dosing period at the following time points: immediately before dosing (Time 0), 20 minutes, 40 minutes, and 1, 1.5, 2, 2.5, 3, 3.5, 4, 4.5, 5, 6, 7, 8, 10, and 12 hours after dosing

Group #1A: (12 Subjects)

| | |
|---|---|
| 1. 20 mg MR tablets | 7 doses (7 days - interconversion) |
| | 7 day washout |
| | safety and PK assessments are performed |
| | for 12 hrs on Day 1 & Day 7 |
| 2. 20 mg MR capsules | 7 doses (7 days - interconversion) |
| | 7 day washout |
| | safety and PK assessments are performed |
| | for 12 hrs on Day 1 & Day 7 |
| 3. Deplin ® (19.5 mg IR | 1 dose (day 29 - interconversion) |
| Tab) - RRP | safety and PK assessments is performed |
| | for 12 hrs. |

End of Group #1A Dosing: 29 Days total.

Group #1B: (12 Subjects)

| | |
|---|---|
| 1. 50 mg MR Capsule | 7 doses (7 days - interconversion) |
| | 7 day washout |
| | safety and PK assessments is performed for |
| | 12 hrs on Day 1 & Day 7 |
| 2. 50 mg MR Tablet | 7 doses (7 days - interconversion) |
| | 7 day washout |
| | safety and PK assessments is performed for |
| | 12 hrs on Day 1 & Day 7 |
| 3. 50 mg IR Tablet - RRP | 1 dose (day 29 - interconversion) |
| | safety and PK assessments is performed for |
| | 12 hr. |

End of Group #1B Dosing: 29 Days total.

### Highlights of PK Single and Multiple Dosing Study:

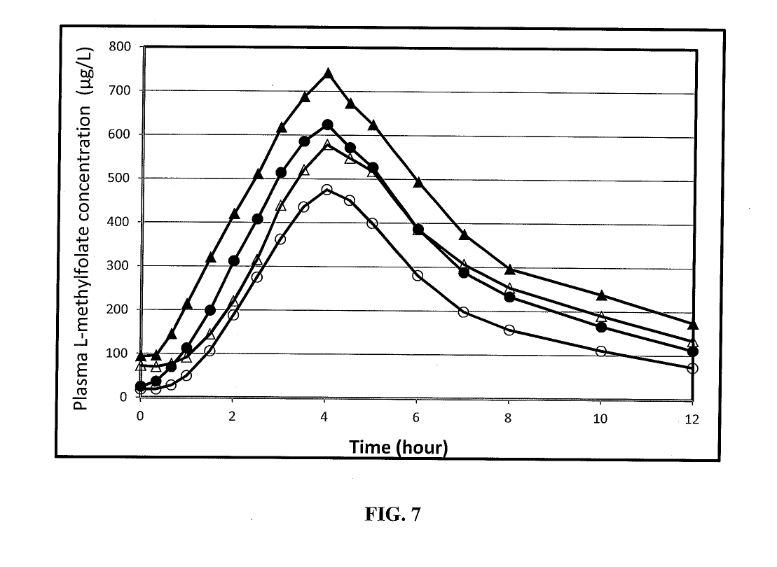

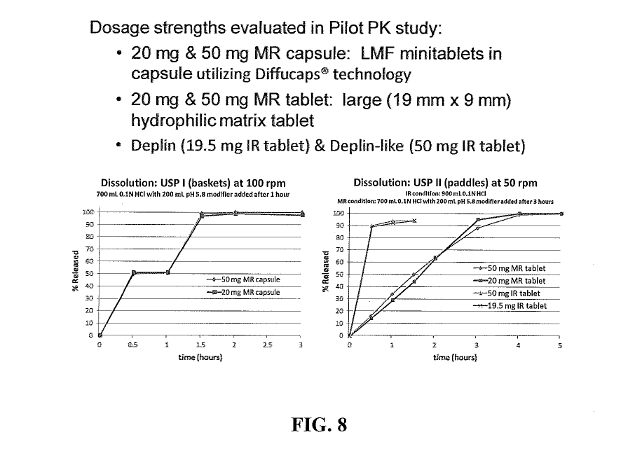

The IR and MR tablet formulations exhibit similar concentration—time profiles upon oral administration although the in vitro drug release profiles are not translated into increased time Tₘₐₓ for Cₘₐₓ for either of the MR formulations (see FIGS. 7 and 8 and Tables 8 and 9).
MR tablets at either dose strength exhibit similar PK profiles upon single dose oral administration in comparison to the corresponding IR tablets.
50 mg MR capsules exhibits PK profiles similar to 50 mg IR tablets and 50 mg MR tablets.
Upon multiple dosing, both Cₘₐₓ and AUC increase significantly for both formulations—MR tablets and MR capsules.

TABLE 9
Summary of L-MTHF PK parameters for 19.5 and 50-mg IR tablets and 20 and 50 mg MR tablets/capsules

| | Tₘₐₓ | Cₘₐₓ | t_{1/2} | AUC₀₋ₗₐₛₜ | AUC_{0-∞} | Tₘₐₓ | Cₘₐₓ | t_{1/2} | AUC₀₋ₗₐₛₜ | AUC_{0-∞} |
|---|---|---|---|---|---|---|---|---|---|---|
| | (hr) | (ng/mL) | (hr) | (ng · hr/mL) | (ng · hr/mL) | (hr) | (ng/mL) | (hr) | (ng · hr/mL) | (ng · hr/mL) |
| | 19.5 mg IR tablets (Deplin) on Day 1 (n = 22) | | | | | 50 mg IR tablets on Day 1 (n = 20) | | | | |
| Mean ± SD | 3.7 ± 0.9 | 495 ± 88 | 4.8 ± 0.9 | 2805 ± 568 | 3453 ± 627 | 3.60 ± 0.5 | 601 ± 117 | 4.9 ± 0.9 | 3725 ± 850 | 4604 ± 1002 |
| | 20 mg MR tablets on Day 1 (n = 22) | | | | | 50 mg MR tablets on Day 1 (n = 20) | | | | |
| Mean ± SD | 4.11 ± 0.87 | 506 ± 60 | 3.9 ± 0.6 | 2654 ± 406 | 3144 ± 530 | 4.15 ± 0.6 | 626 ± 118 | 3.8 ± 0.6 | 3650 ± 765 | 4322 ± 869 |
| | 20 mg MR tablets on Day 7 (n = 22) | | | | | 50 mg MR tablets on Day 7 (n = 21) | | | | |
| Mean ± SD | 4.20 ± 0.7 | 583 ± 62 | 4.4 ± 0.7 | 3330 ± 484 | 4143 ± 591 | 4.3 ± 1.4 | 672 ± 119 | 4.8 ± 0.8 | 4262 ± 815 | 5466 ± 1232 |
| | 20 mg MR capsules on Day 1 (n = 22) | | | | | 50 mg MR capsules on Day 1 (n = 20) | | | | |
| Mean ± SD | 4.25 ± 0.8 | 322 ± 115 | 4.4 ± 0.9 | 1669 ± 623 | 2083 ± 818 | 4.23 ± 1.1 | 496 ± 127 | 5.1 ± 1.7 | 3054 ± 849 | 4050 ± 1249 |
| | 20 mg MR capsules on Day 7 (n = 22) | | | | | 50 mg MR capsules on Day 7 (n = 21) | | | | |
| Mean ± SD | 4.20 ± 0.7 | 362 ± 100 | 5.1 ± 0.9 | 2046 ± 548 | 2705 ± 757 | 4.19 ± 0.54 | 587 ± 103 | 5.2 ± 1.5 | 3860 ± 731 | 5094 ± 1043 |

AUC₀₋ₗₐₛₜ = area under the concentration-time curve from time 0 to time of the last measurable sample after dosing;
AUC_{0-∞} = area under the concentration-time curve from time 0 extrapolated to infinity;
Cₘₐₓ = maximum plasma concentration; SD = standard deviation,
t_{1/2} = terminal elimination half-life; Tₘₐₓ = time of maximum plasma concentration

5.A 50 mg Methylfolate MR Tablets:

A 0.25 ft³ V-blender is charged with (1) approximately half of hypromellose (METOLOSE 90SH), (2) approximately half of micronized L-methylfolate calcium, (3) CARBOPOL 971P, (4) remaining half of L-methylfolate calcium, (5) remaining half of hypromellose after rinsing the methylfolate containing bag (see Table 10 for compositions) and blended for 5 min at 26 rpm to achieve a homogenized pre-blend. The following materials are passed through a Comil equipped with a 062R screen (spacer 0.175″) at 1100 rpm to deagglomerate:

1. approximately half of the mannitol,

2. approximately half of the pre-blend,

3. anhydrous citric acid,

4. silicified microcrystalline cellulose,

5. remaining half of the pre-blend

6. remaining mannitol after rinsing the bag containing the pre-blend.

A 0.5 ft³ V-blender is charged with the Comilled material and blended for 5 minutes. Magnesium stearate is hand screened through a 35 mesh sieve, added into the blender, and further blended for 2 minutes to produce a homogeneously blended compression mix.

TABLE 10
Compositions of MR Tablets

| | 20 mg MR tablets | | 50 mg MR tablets | | 40 mg MR tablets | |
|---|---|---|---|---|---|---|
| Ingredient | mg/tablet | g/batch | mg/tablet | g/batch | mg/tablet | g/batch |
| L-Methylfolate | 24.7 | 185.2 | 61.34 | 460 | 49.1 | 1276 |
| Hypromellose | 72.9 | 546.4 | 70.0 | 525 | 82.4 | 2143 |
| (Metalose 90SH) | | | | | | |
| Silicified MCC | 40.0 | 300.0 | 40.0 | 300 | 40.0 | 1040 |
| (SMCC 90HD) | | | | | | |
| Mannitol | 815.4 | 6115.5 | 773.7 | 5803 | 775.8 | 20170 |
| Carbopol 971P | 7.0 | 52.9 | 15.0 | 112.1 | 12.7 | 329 |
| Citric acid anhydrous | 30.0 | 225.0 | 30.0 | 225 | 30.0 | 780 |
| Magnesium stearate | 10.0 | 75.0 | 10.0 | 75 | 10.0 | 260 |
| Total tablet core | 1000.0 | 7500.0 | 1000.0 | 7500 | 1000.0 | 26000 |
| Opadry II Blue | 30.9 | 370.8 ⁽¹⁾ | 0.31 | 353.4 ⁽¹⁾ | 30.9 | 1004 ⁽¹⁾ |
| | | (231.8) ⁽²⁾ | | (232) ⁽²⁾ | | (804) ⁽²⁾ |
| Carnauba wax | 0.1 | 0.75 | 0.1 | 0.75 | 0.1 | 3 |
| Total tablet weight | 1031 | 7732.5 | 1031 | 7813.0 | 1031 | 26810 |

⁽³⁾ An excess coating solution dispensed to account for process losses. The solids content of the coating solution is 15%.
⁽⁴⁾ Theoretical quantity required.

50 mg L-methylfolate MR tablets are compressed on the Manesty Betapress under the conditions shown in Table 11 below. The process parameters are adjusted so that the tablet properties meet predetermined target values. During the compression run, 15 tablet samples are taken—5 tablets for individual measurement of tablet weight, thickness, 5 tablets for content uniformity testing, and hardness and 5 more as a composite sample for analytical testing. 10 tablets are sampled at the beginning, middle, and end of run for friability testing, and the test data are recorded in the in-process test data sheet.

TABLE 11
Process parameters for tableting of Example 5

| Manesty Beta Press Parameter | | Tablet Parameters - Target with Range | | |
|---|---|---|---|---|
| Tooling (oval shaped | 19 mm | Weight (mg) | 1000 | (970-1030) |
| without embossing) | | | | |
| Speed setting - (rpm) | 25 | Thickness (mm) | 7.6 | |
| Fill Depth (mm) | 11 | Hardness (N) | 170 | (120-220) |

| | | | |
|---|---|---|---|
| Pre-Compression | 6 | Friability - target | 0.3% |
| Force Setting (mm) | | (%) | |
| Main Compression | 5.8 | Appearance | No defects |
| Force Setting (mm) | | | |
| Force Feeder Setting | 5 | | |

A CompuLab Pan Coater is set up with the parameters shown in Table 12 below. The weighed quantity of OPADRY II Blue (4.975 kg) is dissolved/dispersed in 2.819 kg of additional purified water in a stainless steel container while agitating with a low shear agitator. The pan coater is charged with 7.8 kg of MR tablet cores and coated with the stabilizing coating formulation at the process parameters listed in the table below for a weight gain of 3.98% by weight. The weighed quantity of carnauba wax (0.7 g) is added into the product bowl, the pan speed reduced to 5 rpm, and the inlet temperature is set to ‘Off’ to let the tablets to cool down. The tablets are discharged into a double polyethylene bag lined container-closure. After purging the headspace above the bulk tablets with Nitrogen for approximately 1 minute and placing two 500 mL oxygen absorbing packs in direct contact with the bulk tablets and one desiccant pack between the polyethylene bags, the container is closed.

TABLE 12
Process parameters of stabilizing coating

| Parameter | Set up | |
|---|---|---|
| Pan Size: | 24 inches | |
| Number or spray nozzles: | 2 | |
| Nozzle identification: | 2850 | |
| Peristaltic pump: | Masterflex 1 head | |
| Pump tubing size: | Masterflex size 14/16 | |
| Bed to gun distance: | 5-7″ | |
| Inlet air volume - set (CFM): | 255 | |
| Inlet air temp. - target (° C.): | 60 | (50-70) |
| Exhaust air temp. - target (° C.): | 45 | (35-55) |
| Atomizing Air Pressure - set (psi): | 16.5 | (15.5-17.5) |
| Pan speed - set (rpm): | 15 | |
| Pump setting (mL/min) | 24 | (16-32) |
| Product temp. - target (° C.): | 40 | (35-45) |

5.B 40 mg Methylfolate MR Tablets:

A 0.25 ft³ V-blender is charged with (1) approximately half of silicified microcrystalline cellulose (SMCC HD90), (2) micronized L-methylfolate calcium, and (3) remaining silicified microcrystalline cellulose after rinsing the methylfolate containing bag (see Table 3 for compositions) and blended for 5 min at 26 rpm to achieve a homogenized pre-blend. Approximately half of mannitol, the pre-blend, and the remaining mannitol after rinsing the bag containing the pre-blend are sequentially passed through a Comil equipped with a 062R screen (spacer 0.325″) at 1300 rpm to deagglomerate. A 2 ft³ V-blender is charged with the Comilled material, anhydrous citric acid, CARBOPOL 971P, and hypromellose (90SH) and blended at 17 rpm for 8 minutes. The blended material is again passed through the Comil and transferred back into the blender and blended for 16 minutes. Magnesium stearate is hand screened through a 35 mesh sieve, added into the blender, and further blended for 3 minutes to produce a homogeneously blended compression mix. MR tablets weighing one gram are compressed and coated with a stabilizing coating as described above.

1. A stabilized, modified-release pharmaceutical composition comprising a folic acid derivative, and optionally at least one pharmaceutically acceptable excipient.

2. The pharmaceutical composition of claim 1, wherein at least 45% of the folic acid derivative is released within about 1.5 hours when dissolution tested using USP apparatus 2 and two-stage dissolution media (first testing in 700 mL of 0.1N HCl, followed by further testing in pH 5.8 buffer).

3. The pharmaceutical composition of claim 1, wherein the release is effected in proximal small intestine.

4. The pharmaceutical composition of claim 1, wherein the folic acid derivative is selected from the group consisting of methylfolate, tetrahydrofolic acid, dihydrofolic acid, 5-methyltetrahydrofolic acid, 5-formyltetrahydrofolic acid, 10-formyltetrahydrofolic acid, 5,10-methylenetetrahydrofolic acid and 5-formiminotetrahydrofolic acid, or a pharmaceutically acceptable salt thereof.

5. The pharmaceutical composition of claim 4, wherein the folic acid derivative is a pharmaceutically acceptable salt thereof selected from the group consisting of polyglutamate, S-adenosylmethionine salt, glucosamine folate, D-galactosamine folate, D-glucosamine (6S)-tetrahydrofolate, D-galactosamine (6S)-tetrahydrofolate, D-glucosamine 5-methyl-(6S)-tetrahydrofolate.

6. The pharmaceutical composition of claim 1, wherein the folic acid derivative is L-methylfolate calcium salt.

7. The pharmaceutical composition of claim 1, wherein said pharmaceutically acceptable excipient is selected from the group consisting of a hydrophilic dissolution rate controlling matrix forming polymer, hydrophilic dissolution rate controlling coating polymer, plasticizer, bioadhesive polymer, polymeric binder, antioxidant, disintegrant, diluent, sugar, glidant, lubricant or a mixture thereof.

8. The pharmaceutical composition of claim 1, wherein said pharmaceutically acceptable excipient is a diluent selected from the group consisting of dibasic calcium phosphate, calcium sulphate, microcrystalline cellulose, silicified microcrystalline cellulose, and a mixture thereof, and said pharmaceutical composition is in the form of a modified release tablet.

9. The pharmaceutical composition of claim 1, wherein said pharmaceutically acceptable excipient is a sugar selected from the group consisting of a sugar alcohol, mannitol, sorbitol, xylitol or a saccharide, lactose, sucrose, fructose, and a mixture thereof, and said pharmaceutical compositions is in the form of a modified release tablet.

10. The pharmaceutical composition of claim 1, wherein said pharmaceutically acceptable excipient is a polymeric binder selected from the group consisting polyvinylpyrrolidone, hydroxypropylcellulose, or hypromellose, and said pharmaceutical composition is in the form of a modified release tablet.

11. The pharmaceutical composition of claim 1, wherein said pharmaceutically acceptable excipient is a dissolution rate controlling matrix forming polymer selected from the group consisting ethylcellulose, hypromellose, hydroxypropylcellulose, hydroxyethylcellulose, polyethylene oxide, tragcanth gum, alginic acid, alginate, carrageenan, fatty acid, fatty acid ester, and a mixture thereof, and said pharmaceutical composition is in the form of a modified release tablet.

12. The pharmaceutical composition of claim 1, wherein said pharmaceutically acceptable excipient is a hydrophilic, dissolution rate controlling, coating polymer selected from the group consisting of water insoluble ethylcellulose, cellulose acetate, cellulose acetate butyrate, polyvinyl acetate, EUDRAGIT RL or RS polymer; enteric hypromellose phthalate, cellulose acetate phthalate, or polyvinyl acetate phthalate, and a mixture thereof, and said pharmaceutical composition is in the form of a modified release tablet.

13. The pharmaceutical composition of claim 1, wherein said pharmaceutically acceptable excipient is a plasticizer selected from the group consisting of triethyl citrate, diethyl phthalate, glyceryl monostearate, glyceryl triacetate, glyceryl tributyrate, dibutyl phthalate, diethyl phthalate, tributyl citrate, acetyltributyl citrate, triethyl citrate, diethyl sebacate, dibutyl sebacate, polyethylene glycol, vegetable oil, diethyl fumarate, cetyl alcohol ester, castor oil, and a mixture thereof, and said pharmaceutical composition is in the form of a modified release tablet.

14. The pharmaceutical composition of claim 1, wherein said pharmaceutically acceptable excipient is a disintegrant selected from the group consisting of crospovidone, sodium starch glycolate, crosslinked carboxymethyl cellulose sodium, polyethylene oxide, alginic acid, alginate, and a mixture thereof, and said pharmaceutical composition is in the form of a modified release tablet.

15. The pharmaceutical composition of claim 1, wherein said pharmaceutically acceptable excipient is an antioxidant selected from the group consisting of anhydrous citric acid, sodium ascorbate, ascorbic acid, and a mixture thereof, and said pharmaceutical composition is in the form of a modified release tablet.

16. The pharmaceutical composition of claim 1, wherein said pharmaceutically acceptable excipient is a lubricant selected from the group consisting magnesium stearate, stearic acid, glyceryl behenate, sodium stearyl fumarate, and a mixture thereof, and said pharmaceutical composition is in the form of a modified release tablet.

17. The pharmaceutical composition of claim 3, wherein the hydrophilic dissolution rate controlling, matrix forming polymer is selected from the group consisting of low viscosity hydroxypropyl methylcellulose, hydroxypropylcellulose, hydroxyethylcellulose, polyethylene oxide, alginic acid or alginate, and crosslinked carboxymethyl cellulose.

18. The pharmaceutical composition of claim 3, wherein the bioadhesive polymer is selected from the group consisting of crosslinked acrylic acid polymer and hydroxyethylcellulose.

19. The pharmaceutical composition of claim 3, wherein the antioxidant is selected from ascorbic acid and anhydrous citric acid, or a salt thereof.

20. The pharmaceutical composition of claim 3, wherein the polymeric binder is polyvinyl pyrrolidone, hypromellose and hydroxypropylcellulose.

21. The pharmaceutical composition of claim 3, wherein the disintegrant is selected from the group consisting of crospovidone, sodium starch glycolate, crosslinked carboxymethyl cellulose and low-substituted hydroxypropylcellulose.

22. The pharmaceutical composition of claim 3, wherein the diluents is selected from the group consisting of microcrystalline cellulose, silicified microcrystalline cellulose, dipotassium hydrogen phosphate dihydrate, calcium sulfate, lactose, and mannitol, or mixture thereof.

23. A dosage form of the composition of claim 1, wherein the dosage form is a tablet.

24. The dosage form of claim 23 comprising an effective amount of the composition with a hydrophilic dissolution rate controlling polymer, polymeric binder, diluent, and antioxidant.

25. The dosage form of claim 23 comprising an effective amount of the composition with a tow viscosity hydroxypropylmethylcellulose, a crosslinked acrylic acid, spray-dried mannitol, and anhydrous citric acid.

26. The dosage form of claim 23 wherein the constituents are homogeneously blended.

27. The dosage form of claim 23 formed by compressing the constituents into a tablet.

28. The dosage form of claim 23 further comprising a sealant coating.

29. The dosage form of claim 28 wherein the sealant coating is OPADRY II BLUE.

30. The pharmaceutical composition of claim 1, comprising one or more modified-release coated minitablet populations, and optionally immediate release minitablet population, of folic acid derivative, wherein each population comprising at least one pharmaceutically acceptable excipient, and wherein each minitablet population has sealant layer.

31. The pharmaceutical composition of claim 30, wherein at least 80% of the folic acid derivative is released after about 4 hours when dissolution tested using USP apparatus 2 and two-stage dissolution media (first hour testing in 700 mL of 0.1N HCl, followed by further testing in pH 5.8 buffer).

32. The pharmaceutical composition of claim 30, comprising a modified-release coated minitablet population and an immediate release minitablet population wherein the ratio of modified-release coated minitablet population to immediate release minitablet population is from about 90:10 to about 50:50.

33. The pharmaceutical composition of claim 30, wherein the modified-release coated minitablet population provides for a sustained release or timed, pulsatile release.

34. The pharmaceutical composition of claim 33, wherein the modified-release coated minitablet population providing for a sustained release has a coating comprising a water-insoluble polymer alone or a water-insoluble polymer in combination with a water soluble polymer at a ratio of from about 9:1 to about 5:5 for a coating weight gain of from about 1 to about 10% w/w.

35. The pharmaceutical composition of claim 33, wherein modified-release coated minitablet population providing for a timed, pulsatile release has a coating comprising a water-insoluble polymer in combination with an enteric polymer at a ratio of from about 9:1 to about 1:3 for a coating weight gain of from about 1 to about 10% w/w.

36. The pharmaceutical composition of claim 35, wherein the modified-release coated minitablet population providing for a timed, pulsatile release, in addition to having a coating comprising a water-insoluble polymer in combination with an enteric polymer, further comprises an enteric polymer coating contributing a coating weight gain of from about 10 to about 30% w/w, and wherein the timed, pulsatile release and enteric coatings can be coated in either order.

37. The pharmaceutical composition of claim 36, wherein said enteric polymer coating is selected from the group consisting of cellulose acetate phthalate, hydroxypropyl methylcellulose phthalate, hydroxypropyl methylcellulose succinate, polyvinyl acetate phthalate, pH-sensitive methacrylic acid-methylmethacrylate copolymers and shellac, or a mixture thereof.

38. The pharmaceutical composition of claim 34, wherein said water-insoluble polymer is selected from the group consisting of ethylcellulose, cellulose acetate, cellulose acetate butyrate, polyvinyl acetate and neutral methacrylic acid-methylmethacrylate copolymers, or a mixture thereof.

39. The pharmaceutical composition of claim 30, wherein said modified-release membrane-coating of said modified-release coated minitablet population further comprises a plasticizer selected from the group consisting of triacetin, tributyl citrate, tri-ethyl citrate, acetyl tri-n-butyl citrate, diethyl phthalate, dibutyl sebacate, polyethylene glycol, polypropylene glycol, castor oil, acetylated mono glyceride and acetylated diglyceride, or mixture thereof.

40. The pharmaceutical composition of claim 30, said modified-release membrane-coating of said modified-release coated minitablet population further comprises an anti-tack agent selected from the group consisting of talc, stearic acid, sodium stearyl fumarate and magnesium stearate, or mixture thereof.

41. The pharmaceutical composition of claim 30 wherein the immediate-release minitablet population comprises L-methylfolate, hydroxypropyl methylcellulose, silicified microcrystalline cellulose, dibasic calcium phosphate dihydrate, sodium starch glycolate, and anhydrous citric acid, and magnesium stearate.

42. The pharmaceutical composition of claim 1 wherein the folic acid derivative in amounts sufficient to provide a pharmacokinetic profile suitable for a once- or twice-daily dosing regimen in patients in need thereof.

43. The pharmaceutical composition of claim 30, comprising
a) immediate release minitablet population; and
b) modified-release coated minitablet population that provides for timed, pulsatile release through coating comprising a water-insoluble polymer in combination with an enteric polymer, disposed over an immediate-release minitablet population; and
c) the immediate-release minitablet cores comprise a stabilizing membrane-coating disposed over minitablet cores at a weight gain of from 2 to about 5% w/w;
wherein the drug release peaks of the immediate-release and timed pulsatile release minitablet populations are separated by about 1-2 hours when dissolution tested using USP apparatus 2 in 700 mL of 0.1N HCl for one hour followed by testing in 900 mL of pH 5.8 buffer for an additional 3 hours.

44. A method for the preparation of the modified-release coated minitablet population of claim 30, comprising:
a) preparing immediate release minitablet population comprising a folic acid derivative and at least one pharmaceutically acceptable excipient, and optionally providing a sealant membrane-coating onto the immediate release minitablet population; and
b1) coating the immediate release minitablet population from step a) with a modified-release membrane-coating comprising an enteric polymer for a weight gain of from about 10% to about 30% w/w to yield a enteric coated modified-release minitablet population; or
b2) coating the immediate release minitablet population of step (a) or enteric coated modified-release minitablet population of step (b) with a timed pulsatile release coating comprising a water-insoluble polymer in combination with an enteric polymer at a ratio of from about 9:1 to 1:3 for a combined weight gain of from about 5% to about 10% w/w to yield either a timed pulsatile release coated modified-release minitablet population or a timed pulsatile release, enteric coated modified-release minitablet population.

45. A method for the preparation of modified release pharmaceutical dosage form comprising the composition of claim 1, comprising
a) preparing a blend comprising a folic acid derivative and at least one pharmaceutically acceptable excipient wherein the folic acid derivative is homogeneously dispersed in the blend;
b) optionally further blending the blend from step (a) with a pharmaceutically, acceptable lubricant;
c) compressing the blend from step (a) or (b) into a tablet using a rotary tablet press; and
d) optionally applying a sealant coating onto tablet from step (c).

46. A modified release pharmaceutical dosage form comprising the composition of claim 1, provides a mean maximum plasma concentration Cₘₐₓ within the range of about 80% to 125% of about 700 ng/mL, and an AUC_{0-24 hr} within the range of about 80% to 125% of about 5000 ng·hr/mL, following oral administration.

47. A method of treating a patient subject to major depressive disorder, diabetic peripheral neuropathy, dysthymia, schizophrenia, or degenerative dementia of the Alzheimer type comprising administering a therapeutically effective amount of the pharmaceutical composition of claim 1 to said patient.

48. The pharmaceutical composition of claim 35, wherein said water-insoluble polymer is selected from the group consisting of ethylcellulose, cellulose acetate, cellulose acetate butyrate, polyvinyl acetate and neutral methacrylic acid-methylmethacrylate copolymers, or a mixture thereof.
